(19) United States
(12) Patent Application Publication
(10) Pub. No.: US 2005/0026223 A1
(43) Pub. Date: Feb. 3, 2005

(54) Method and compositions for increasing bone mass

(75) Inventors: Stavros C. Manolagas, Little Rock, AR (US); Robert L. Jilka, Little Rock, AR (US); Robert S. Weinstein, Little Rock, AR (US); Teresita Bellido, Little Rock, AR (US); Donald Bodenner, Little Rock, AR (US); Stavroula Kousteni, Little Rock, AR (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Board of Trustees of the University of Arkansas, Little Rock, AR (US)

(21) Appl. No.: 10/829,028

(22) Filed: Apr. 21, 2004

Related U.S. Application Data
(63) Continuation of application No. 09/414,091, filed on Oct. 7, 1999, now abandoned.
(60) Provisional application No. 60/151,486, filed on Aug. 30, 1999.
(60) Provisional application No. 60/136,260, filed on May 27, 1999.
(60) Provisional application No. 60/119,080, filed on Feb. 8, 1999.

(51) Int. Cl. G01N 33/53; G01N 33/567; A61K 31/56

(52) U.S. Cl. 435/7.2; 514/169

(57) ABSTRACT

The invention as disclosed provides a method to increase bone mass without compromising bone strength or quality, through the administration to a host of a compound that binds to the estrogen or androgen receptor without causing hormonal transcriptional activation.

### RELATED APPLICATION

This application is a continuation of U.S. application Ser. No. 09/414,091, filed Oct. 7, 1999, which claims the benefit of U.S. Provisional Application No. 60/151,486 filed Aug. 30, 1999, U.S. Provisional Application No. 60/136,260 filed May 27, 1999 and U.S. Provisional Application No. 60/119,080 filed Feb. 8, 1999. The entire teachings of the above applications are incorporated herein by reference.

### Federal Funding

This invention was funded in part through a grant from the National Institutes of Health. Therefore, the federal government has certain rights in this invention.

### BACKGROUND OF THE INVENTION

1. Field of the Invention

This invention is in the field of bone physiology, and in particular provides methods and compositions that include compounds to increase bone mass, i.e., to achieve bone anabolism. The compounds bind to the estrogen or androgen receptor without causing significant hormonal transcriptional activation.

2. Description of the Related Art

Bones consist of living cells embedded within a matrix of proteins and minerals. Bones provide support and protection to the vital organs of the animal, and give strength and form to its structure.

Osteoporosis is a decrease in bone mass in combination with microarchitectural deterioration which leads to bone fragility and fractures. Treatments for osteoporosis have historically focused on the prevention of further bone loss. In contrast, a bone anabolic agent is one that substantially increases bone mass. To date, while there have been several drugs approved by the U.S. Food and Drug Administration for the treatment of osteoporosis, it is believed that no drug has yet been approved in the United States to be used as a bone anabolic agent, for either humans or other animals. Bone is a dynamic tissue which undergoes continual resorption and formation through a remodeling process, which is accomplished by two types of cells: osteoclasts, which erode cavities, and osteoblasts that synthesize new bone matrix. Remodeling takes place mainly on the internal surfaces of bone and it is carried out not by individual cells, but rather by temporary anatomical structures, termed basic multi-cellular units (BMUs), comprising teams of osteoclasts in the front and osteoblasts in the rear. In an established BMU, bone resorption and formation happens at the same time.

After osteoclasts stop resorbing bone, they die by apoptosis and are quickly removed by phagocytes. During the longer lifespan of the osteoblasts (about three months, as compared to three weeks for osteoclasts), some osteoblasts convert to lining cells that cover quiescent bone surfaces and some are entombed within the mineralized matrix as osteocytes (Parfitt, In: Bone, Telford and CRC Press, PP351-429, 1990). However, the majority (65%) of osteoblasts that originally assembled at the remodeling site die by apoptosis (Jilka et al, JBMR 13:793-802, 1998).

Most metabolic disorders of the adult skeleton result from an imbalance between the resorption of old bone by osteoclasts and its subsequent replacement by osteoblasts. Changes in cell numbers, as opposed to individual cell activity (Manolagas and Jilka, NEJM 332:305-311, 1995), appear to be the cause of most metabolic bone diseases, including the three most common forms of osteoporosis: osteoporosis due to sex steroid deficiency in females and males (Jilka et al., Science 257:88-91, 1992; Jilka et al., JCI 101:1942-1950, 1998; Bellido et al., JCI 95:2886-2895, 1995; Weinstein et al., Endocrinology 138:4013-4021, 1997); osteoporosis due to old age (Jilka et al., JCI 97:1732-1740, 1996); and osteoporosis due to glucocorticoid-excess (Weinstein et al., JCI 102:274-282, 1998; Weinstein et al, Bone, 23:S461, 1998; Bellido et al, Bone, 23:S324, 1998).

Agents that reduce bone turnover by inhibiting the activation of bone remodeling (commonly but inaccurately referred to as “antiresorptive”) increase bone mass by a maximum of 6-10%, and more typically, 2-3%, as measured by Dual Energy X-Ray absorptometry (DEXA). Most of this increase is in the first 1-2 years and is due to contraction of the remodeling space. Modest further increases may result from more complete secondary mineralization. Improvement of focal balance due to reduction of resorption depth has been demonstrated in animal experiments, but not yet in human subjects. Regardless of the mechanism, an increase of less than 10% will in almost all cases fail to restore bone mass to its peak value and fail to reestablish trabecular connectivity so that fracture risk will remain increased.

There are a wide variety of needs for bone anabolic agents for humans as well as animals. Examples of uses for bone anabolic agents in humans, besides patients with osteoporosis, include the strengthening of bone in healthy subjects who engage in strenuous physical activities such as sports or manual labor, and the strengthening of bone in persons who do not have osteoporosis but might be subject to osteoporosis in the future because the person is in a risk group for that disease. Other uses for a bone anabolic agent in humans include the treatment of persons who fail to obtain an adequate bone mass at the completion of growth or persons who are born with unusually fragile bones, persons who have a genetic predisposition to a bone catabolic disease, or an orthopedic bone disease such as joint degeneration, non-union fractures, orthopedic problems caused by diabetes, periimplantitis, poor responses to bone grafts, implants, fracture.

Likewise, there are many uses for bone anabolic agents in animals. For example, it would be useful to increase the bone mass in horses and dogs used for labor as well as those used in sports such as racing. It would also be useful to increase the bone mass in chickens and turkeys used in meat production to maximize the amount of meat yield per animal.

There are currently ten classes of drugs that are used in the treatment of osteoporosis: anabolic steroids, bisphosphonates, calcitonins, estrogens/progestogens, Selective Estrogen Receptor Modulators (SERMs) such as raloxifene, phytoestrogen, parathyroid hormone (“PTH”), fluoride, Vitamin D metabolites, and calcium preparations. No compound within these classes has been approved as a bone anabolic agent.

Anabolic Steroids (Androgens)

Anabolic steroids (androgens) have been known to build muscle mass in the host. However, there has been no reported evidence that they function as bone anabolic agents as defined herein (Snyder et al, JCEM 84:1966-1972, 1999). Androgens are typically used as a replacement therapy for male hypogonadal disorders and they are used in adolescent males with a history of delayed puberty or growth. Androgens can produce significant side effects when taken over a period of time, including water retention, jaundice, decreased high density lipoprotein and increased low density lipoprotein, hepatic toxicity (most usually associated with the 17α-alkylated androgens), hepatic carcinoma, increased risk of cardiovascular disease, and when taken in large dosages, irrationality, psychotic episodes, violent behavior, and death. U.S. Pat. No. 5,565,444 discloses the use of an androgen for the treatment of bone loss or for increasing bone mass.

Calcitonin

Endogenous calcitonin is a polypeptide hormone involved in the regulation of calcium and bone metabolism. Forms used therapeutically include calcitonin (pork), extracted from pig thyroid, a synthetic human calcitonin; elcatonin, a synthetic analogue of eel calcitonin; and salcatonin, a synthetic salmon calcitonin. They all have the property of lowering plasma-calcium concentration by diminishing the rate of bone resorption. Calcitonins are typically administered subcutaneously or by intramuscular injection.

Bisphosphonates

Bisphosphonates have been widely used to treat osteoporosis. The bisphosphonate disodium etidronate has similar effects on bone mass and fractures in established osteoporosis to those of calcitonin, but cannot be given for a prolonged period because of the risk of osteomalacia. Bisphosphonate alendronate treatment at a dose of 10 mg/day results in a 5% increase in spinal bone mineral density (BMD) over the first year (Dempster, Exploiting and Bypassing the Bone Remodeling Cycle to Optimize the Treatment of Osteoporosis, Journal of Bone and Mineral Research, Volume 12, Number 8, 1997, pages 1152-1154). BMD continues to increase, albeit at a slower rate, at this site during the second and third years of treatment. The magnitude and duration of the increase in BMD has led to speculation that alendronate is doing more than simply reducing remodeling space and that it may possess anabolic activity. The bisphosphonate etidronate reduced resorption depth in human iliac trabecular bone by almost 30% after one year of treatment, but no such data are yet available for alendronate. Etidronate did not change the thickness of trabecular packets, but recent studies in osteoporotic women suggest that this is increased after two years of alendronate treatment at 10 and 20 mg/day. This result was not confirmed after three years of treatment.

In another article, Dempster (Dempster D. W., New concepts in bone remodeling, In: Dynamics of Bone and Cartilage Metabolism, Chapter 18, pp.261-273, Acad. Press, 1999) confirms that the potential for an agent that can increase bone mass and hence reverse the skeletal defect in patients with osteoporosis is great, particularly if in doing so it also repairs microarchitectural damage. He notes that estrogens and calcitonin primarily stabilize bone mass and prevent further loss of bone, although a transient small increment in mass is often reported, particularly in patients with elevated levels of bone remodeling. Dempster et al conclude that this is not a true anabolic effect but is related to the temporal effects on turnover in which resorption declines initially followed by a reduction in formation that may take several months.

Albeit, bisphosphonates have anti-apoptotic effects on osteoblasts and osteocytes (Plotkin et al. Bone, 23:S157, 1998). Significantly, the anti-apoptotic effect of bisphosphonates in vitro is achieved with doses 100-1000 lower than the doses at which these same agents inhibit osteoclast activity; and additionally can be demonstrated with bisphosphonates that do not block osteoclast activity at all (compound IG9204). U.S. Pat. No. 4,870,063 discloses a bisphosphonic acid derivative to increase bone mass. U.S. Pat. Nos. 5,532,226 and 5,300,687 describe the use of trifluoromethylbenzylphosphonates to increase bone mass. U.S. Pat. No. 5,885,973 to Papapoulos, et al, discloses a bone mass anabolic composition that includes olpandronate, which is a bisphosphonate.

Estrogens/progestogens

Estrogens/progestogens (anti-remodeling and anti-resorptive compounds) as a class have not to date been shown to increase bone mass by more than 10%, but instead have been used to retard the effect of osteoporosis. Estrogens are currently the most effective method of preventing osteoporosis in postmenopausal women.

U.S. Pat. No. 5,183,815 discloses the use of a steroidal hormone covalently linked to a hydroxy alkyl-1,1-bisphosphonate. U.S. Pat. No. 5,843,934 claims that an estrogen having insubstantial sex-related activity can be administered to a patient to retard the adverse effects of osteoporosis in a male or female. The '934 patent does not address how to select a compound to increase bone mass, but instead teaches how to retard the effect of bone loss. WO 98/22113 filed by the University of Florida Research Foundation, Inc. discloses methods to utilize an isomer of an estrogen compound to confer cytoprotection on a population of cells associated with an ischemic event.

Phytoestrogens

Little is known about the actions of phytoestrogens on bone (Fitzpatrick, L. A., Mayo Clinic Proceedings, 74:601-607, 1999). Soy protein did not prevent increased bone turnover in cynomolgus monkeys; they actually increased it. However, BMD declined after two years in postmenopausal women taking only calcium but did not change in those receiving ipriflavone. Isoflavone significantly increased spinal BMD in postmenopausal women after 6 months of 40 mg/day of soy protein supplementation (containing 90 mg isoflavones) but not with lower doses (56 mg/day) (Feinkel, E Lancet, 352:762, 1998).

Parathyroid Hormone (PTH)

Daily injections of parathyroid hormone (PTH), an agent known for its role in calcium homeostasis, increases bone mass in animals and humans, as does the related PTH-related hormone PHTrP, the only other known ligand of the PTH receptor. Whereas increased prevalence of apoptosis of osteoblasts and osteocytes are key pathogenic mechanisms for osteoporosis (Weinstein et al., J Clin Invest, 102:274-282, 1998; Weinstein et al, Bone, 23:S461, 1998; Bellido et al, Bone, 23:S324, 1998), the reverse, i.e., postponement of osteoblast apoptosis, is the principal, if not the sole, mechanism for the anabolic effects of intermittent parathyroid hormone administration on bone (Jilka et al., J. Clin. Invest. 104:439-446, 1999). The increased bone mineral density, osteoblast perimeter and bone formation rate that occur with intermittent PTH administration in mice happen without a change in osteoblast production. Instead, the anabolic effect of the drug is due to decreased prevalence of osteoblast apoptosis from 1.7-2.2% to as little as 0.1-0.4%, while the osteocytes in the newly made lamellar cancellous bone are closer together and more numerous than those found in the animals receiving vehicle alone. The closely spaced, more numerous osteocytes are the predictable consequence of protecting osteoblasts from apoptosis. The anti-apoptotic effect of PTH on osteoblasts as well as osteocytes has been confirmed in vitro using primary bone cell cultures and established cell lines.

The use of teriparatide (the 1-34 amino acid fragment of human parathyroid growth hormone) to stimulate bone formation has also been investigated; teriparatide administered as daily injections has been reported to selectively increase the trabecular bone density of the spine in osteoporotic patients.

U.S. Pat. No. 5,510,370 discloses the use of a combination of PTH and raloxifene to increase bone mass. U.S. Pat. No. 4,833,125 discloses the use of PTH in combination with either a hydroxylated vitamin D derivative, or a dietary calcium supplement.

Calcium Preparations

Calcium preparations, while useful as a dietary supplement for persons who are calcium deficient, have not been shown effective to increase bone mass. However, they may reduce the rate of bone loss. U.S. Pat. No. 5,618,549 (a calcium salt) describes the use of calcium.

Fluoride

The most thoroughly studied anabolic agent, sodium fluoride, can increase vertebral bone mass by 10% a year for at least four years but there is controversy about the quality of the bone formed. Sodium fluoride has not been approved as a bone anabolic agent. It has been difficult to establish anti-fracture efficacy because of serious qualitative abnormalities. First, much of the new bone is initially woven rather than lamellar. Second and more important, there is severe impairment of bone mineralization, in spite of sodium fluoride's effectiveness in increasing bone mass.

U.S. Pat. No. 5,071,655 discloses a composition to increase bone mass that includes a fluoride source and a mitogenic hydantoin.

SERMs

SERMs such as tamoxifen and raloxifene have also been used to treat osteoporosis. A recent study carried out with raloxifene indicated that after three years of treatment, women on raloxifene had 30-50% fewer spinal fractures, and had 2-3% increase in bone density in their hips and spine, but showed no fewer nonspinal fractures, a category that includes hip fractures (Ettinger, B., JAMA, 282:637-645, 1999).

U.S. Pat. No. 4,970,237 discloses the use of clomiphene to increase bone mass in premenopausal women.

Vitamin D Derivatives

There have been conflicting reports about the value of Vitamin D or its derivatives on bone loss and bone anabolism. Some studies on the hormonal metabolite of vitamin D, calcitriol, have reported an increase in spinal bone density, but others have found no effect.

The following patents describe the use of Vitamin D derivatives to treat bone disease: U.S. Pat. Nos. 4,973,584; 5,750,746; 5,593,833; 5,532,391; 5,414,098; 5,403,831; 5,260,290; 5,104,864; 5,001,118; 4,973,584; 4,619,920; and 4,588,716.

Other Compounds

The following patents disclose the use of other compounds for the treatment of bone disease: U.S. Pat. Nos. 5,753,649 and 5,593,988 (azepine derivative); U.S. Pat. No. 5,674,844 (morphogen); U.S. Pat. No. 5,663,195 (cyclooxygenase-2 inhibitor); U.S. Pat. No. 5,604,259 (ibuprofen or flurbiprofen); U.S. Pat. No. 5,354,773 (bafilomycine); U.S. Pat. No. 5,208,219 (activin); U.S. Pat. No. 5,164,368 (growth hormone releasing factor); and U.S. Pat. Nos. 5,118,667, 4,870,054 and 4,710,382 (administration of a bone growth factor and an inhibitor of bone resorption).

U.S. Pat. No. 5,859,001 discloses the use of non-estrogen compounds having a terminal phenol group in a four-ring cyclopentanophenanthrene compound structure to confer neuroprotection to cells.

U.S. Pat. No. 5,824,672 discloses a method for preserving tissues during transplantation procedures that includes administering an effective dose of a cyclopentanophenanthrene compound having a terminal phenol A ring.

WO 98/31381 filed by the University of Florida Research Foundation, Inc. discloses a method for enhancing the cytoprotective effect of polycyclic phenolic compounds on a population of cells that involves the steps of administering a combination of polycyclic phenolic compounds and anti-oxidants to achieve an enhanced effect. One disclosed combination is glutathione and estrogen.

It is an object of the present invention to provide a method to increase bone mass in a host by at least 10% per year without a loss in bone strength (defined by fracture incidence in vivo and mechanical strength in vitro) and/or deterioration of bone quality (as defined by abnormal collagen orientation and excessive accumulation of unmineralized bone matrix, determined, for example, with histomorphometry).

It is another object of the present invention to provide a method to rebuild strong bones instead of preventing further loss of bone.

It is a further object of the present invention to provide a method to select compounds that increase bone mass in a host at least 10% per year without a loss in bone strength or quality.

It is a still further object of the present invention to provide a method to increase bone strength by at least 20%.

### SUMMARY OF THE INVENTION

In a first embodiment, a method for increasing bone mass in a host at least 10% without a loss in bone strength or quality is provided that includes administering an effective amount of a compound that (i) binds to the estrogen α or β receptor (or the equivalent receptor in the host animal) with an association constant of at least 10⁸ M⁻¹ , and preferably, at least 10¹⁰ M⁻¹ : (ii) (a) induces estrogenic gene transcriptional activity at a level that is no greater than 10% that of 17β-estradiol, and preferably no greater than 5, 1 or even 0.1% that of 17β-estradiol when administered in vivo at a dosage of at least 0.1 ng/kg body weight or in vitro in osteoblastic or osteocytic cells with natural estrogen receptors or cells transfected with estrogen receptors or (b) induces an increase in uterine weight of no more than 10% that of 17β-estradiol (or the equivalent compound in a host animal); (iii) induces the phosphorylation of extracellular signal regulated kinase (ERK) when administered in vivo at a dosage of at least 0.1 ng/kg body weight or in vitro in osteoblastic cells with natural estrogen receptors or cells transfected with estrogen receptors; and (iv) has an anti-apoptotic effect on osteoblasts and osteocytes at an in vivo dosage of at least 0.1 ng/kg body weight or in vitro in osteoblastic or osteocytic cells with natural estrogen receptors or cells transfected with the estrogen receptor. In another aspect of this first embodiment of this invention, the compound is not an estrogen compound, as that term is defined below. In yet another aspect of this first embodiment, the compound is an estrogen compound which is converted to a nonestrogen by attaching a substituent which prevents the compound from entering the cell but does not significantly affect the binding of the compound to the estrogen cell-surface receptor.

In a second embodiment, a method for increasing bone mass in a host at least 10% without a loss in bone strength or quality is provided that includes administering an effective amount of a compound that (i) binds to the androgen receptor (or the equivalent receptor in the host animal) with an association constant of at least 10⁸ M⁻¹ , and preferably, at least 10¹⁰ M⁻¹ : (ii) (a) induces androgenic gene transcriptional activity at a level that is no greater than 10% that of testosterone, and preferably no greater than 5, 1 or even 0.1% that of testosterone when administered in vivo at a dosage of at least 0.1 ng/kg body weight or in vitro in osteoblastic cells with the natural androgen receptor or cells transfected with the androgen receptor or (b) induces an increase in muscle weight of no more than 10% that which is induced by testosterone (or the equivalent compound in a host animal); (iii) induces the phosphorylation of extracellular signal regulated kinase (ERK) when administered in vivo at a dosage of at least 0.1 ng/kg body weight or in vitro in osteoblastic cells with the natural androgen receptor or cells transfected with the androgen receptor; and (iv) has an anti-apoptotic effect on osteoblasts and osteocytes at an in vivo dosage of at least 0.1 ng/kg body weight or in vitro in osteoblastic cells with the natural androgen receptor or transfected with the androgen receptor. In another aspect of the second embodiment, the compound is not an androgen. In yet another aspect of this second embodiment, the compound is an androgen compound which is converted to a nonandrogen by attaching a substituent which prevents the compound from entering the cell but which does not significantly affect the ability of the compound to bind to the androgen cell-surface receptor.

In other aspects of the first or second embodiment of this invention, the compound has a pro-apoptotic effect on osteoclasts at an in vivo dosage of at least 0.1 ng/kg body weight, or in osteoclastic cells with natural estrogen receptors or cells transfected with estrogen receptors.

The disclosed invention is based on the fundamental discovery that bone loss occurs because of an increase in osteoblast and perhaps osteocyte apoptosis, which can be inhibited by a compound that binds to an estrogen or androgen receptor, which induces the phosphorylation of ERKs without significant hormonal transcriptional activation. The discovery of this fundamental pathway allows the selection of compounds which provide a maximum effect on bone mass and strength.

Therefore, in a third embodiment, a method for selecting a compound that increases bone mass in a host at least 10% without a loss in bone strength or quality is provided that includes evaluating whether the compound (i) binds to the estrogen or androgen receptor (or the equivalent receptor in the host animal) with an association constant of at least 10⁸ M⁻¹ , and preferably, at least 10¹⁰ M⁻¹ : (ii) (a) induces estrogenic or androgenic gene transcriptional activity at a level that is no greater than 10% that of 17β-estradiol or testosterone, and preferably no greater than 5, 1 or even 0.1% that of 17β-estradiol or testosterone, as appropriate, when administered in vivo at a dosage of at least 0.1 ng/kg body weight or in vitro in osteoblastic cells with the natural androgen or estrogen receptor or cells transfected with the androgen or estrogen receptor or (b) induces an increase in uterine weight of no more than 10% that which is induced by 17β-estradiol or muscle weight of no more than 10% that which is induced by testosterone (or the equivalent compound in a host animal); (iii) induces the phosphorylation of extracellular signal regulated kinase (ERK) when administered in vivo at a dosage of at least 0.1 ng/kg body weight or in vitro in osteoblastic or osteocytic cells with the natural androgen or estrogen receptor or cells transfected with the androgen or estrogen receptor; and (iv) has an anti-apoptotic effect on osteoblasts and osteocytes at an in vivo dosage of at least 0.1 ng/kg body weight or in vitro in osteoblastic and osteocytic cells with the natural androgen or estrogen receptor or cells transfected with the androgen or estrogen receptor.

Estrogenic compounds like 17α-estradiol and synthetic polycyclic phenols, such as estratriene-3-ol inhibit osteoblast and osteocyte apoptosis in vitro. Yet unlike the classical mechanism of estrogen receptor action that involves direct or indirect interaction with the transcriptional apparatus, the receptor-dependent anti-apoptotic effects of these compounds are nongenomic, as they are due to rapid (within 5 minutes) phosphorylation of ERKs. Estratriene-3-ol increases bone mass in both estrogen-replete and estrogen-deficient mice. Estratriene-3-ol, when given in low doses, has little effect on estrogenic-type activity but also has little effect on bone mass. As the dosage increases, both effects increase. To optimize the use of this compound or others exhibiting this type of activity, one can derivatize the compound to preserve the estrogen-binding activity and decrease the transcriptional activity as described in detail herein, including by attaching a substituent or moiety that inhibits cell penetration.

Compounds selected according to the criteria provided herein can also be used for the augmentation of bone mass and/or fracture prevention in diseases characterized by low bone mass and increased fragility. The compounds can also be used to treat bone disease states in which osteoblastogenesis is decreased, such as senile osteoporosis, and glucocorticoid-induced osteoporosis—especially in growing children and adolescents, during which time in whom interfering with bone remodeling is detrimental.

### BRIEF DESCRIPTION OF THE DRAWINGS

The Figures provided herein illustrate embodiments of the invention and are not intended to limit the scope of the invention.

FIG. 1 provides nonlimiting examples of one class of compounds that can be used to increase bone mass without adversely affecting bone strength.

FIG. 2 is a bar chart graph of the degree of apoptosis of osteoblasts and osteocytes in murine vertebral bone as a function of estrogen deficiency. Swiss Webster mice (four months old) were ovariectomized. Twenty eight days later, the animals were sacrificed, vertebrae were isolated, fixed and embedded, and then undecalcified in methacrylate. The prevalence of osteoblast and osteocyte apoptosis was determined by the TUNEL method with CuSO₄ enhancement, and was found to be dramatically increased following loss of estrogen. ***P<0.00001; *P<0.0382.

FIG. 3 is a series of bar chart graphs which illustrate the percentage of Etoposide-induced osteoblast apoptosis versus the log of the concentration of added estrogens 17β-estradiol, 17β-estradiol-BSA, 17α-estradiol, and estratriene-3-ol. Osteoblastic cells derived from murine calvaria were pretreated with the sterols for 1 hour before the addition of the pro-apoptotic agent, etoposide. Apoptosis was determined after 6 hours by trypan blue uptake (Jilka et al, J. Bone and Min. Res. 13:793:802, 1998). * indicates p<0.05 versus etoposide alone, by analysis of variance (ANOVA) (Student-Newman-Keuls method).

FIG. 4 is a series of bar chart graphs of the inhibition of etoposide-induced apoptosis of osteocytes (MLO-Y4) by 17β-estradiol, 17β-estradiol-BSA, 17α-estradiol, and estratriene-3-ol. Cells were pretreated with the indicated concentrations of the compounds for 1 hour before the addition of the pro-apoptotic agent etoposide. Apoptosis was determined after 6 hour by trypan blue uptake as described in FIG. 3. * indicates p<0.05 versus etoposide alone, by ANOVA (Student-Newman-Keuls method).

FIG. 5 is a series of bar chart graphs that indicates that the anti-apoptotic effect of 17β-estradiol, 17β-estradiol-BSA, 17α-estradiol, and estratriene-3-ol (E-3-ol) on etoposide-induced apoptosis of osteoblasts is abrogated by the estrogen receptor antagonist, ICI182,780. Osteoblastic cells derived from murine calvaria were pretreated for 1 hour with the pure receptor antagonist ICI182,780 (10⁻⁷ M) before the addition of the test agents (10⁻⁸ M). Apoptosis was induced and quantified as described in FIG. 3. * indicates p<0.05 versus etoposide alone, by ANOVA (Student-Newman-Keuls method).

FIG. 6 is a series of bar chart graphs that indicates that the anti-apoptotic effect of 17β-estradiol, 17β-estradiol-BSA, 17α-estradiol, and estratriene-3-ol (E-3-ol) on MLO-Y4 osteocytic cells is abrogated by the estrogen receptor antagonist, ICI182,780. MLO-Y4 cells were pretreated for 1 hour with the pure receptor antagonist ICI182,780 (10⁻⁷ M) before the addition of the test agents (10⁻⁸ M). Apoptosis was induced and quantified as described in FIG. 3. * indicates p<0.05 versus etoposide alone, by ANOVA (Student-Newman-Keuls method).

FIG. 7 is a series of bar chart graphs which demonstrate that estrogen receptor α or β is required for the anti-apoptotic effects of 17β-estradiol, 17α-estradiol, and estratriene-3-ol on the etoposide-induced apoptosis of osteoblasts. CMV promoter alone and CMV promoter-driven cDNA for mERα or mErβ were stably transfected into HeLa cells. Subconfluent cultures were treated for 1 hr with 10⁻⁸ M 17α-estradiol, 17β-estradiol, or estratriene-3-ol followed by a 6 hr incubation with etoposide (5×10⁻⁵ M). Cells were trypsinized, pelleted and trypan blue positive cells enumerated. Each bar represents mean of duplicate experiments ±SEM. *P<0.02 versus etoposide alone.

FIG. 8 is Western blot which demonstrates that 17β-estradiol, 17α-estradiol, 17β-estradiol-BSA or estratriene-3-ol activate the extracellular signal regulated kinases (ERKs). MLO-Y4 osteocytic cells were incubated for 25 minutes in serum-free medium. Subsequently, 17β-estradiol, 17α-estradiol, 17β-estradiol-BSA or estratriene-3-ol (10⁻⁸ M) were added and cells incubated for an additional 5, 15, or 30 min. Cell lysates were prepared and proteins were separated by electrophoresis in polyacrylamide gels and transferred to PVDF membranes. Western blotting was performed using a specific antibody recognizing phosphorylated ERKs 1 and 2, followed by reblotting with an antibody recognizing total ERKs. Blots were developed by enhanced chemiluminescence.

FIG. 9 is a Western blot which demonstrates that the effect of estrogenic compounds on the activation of ERK1/2 is blocked by the specific inhibitor of ERK kinase, PD98059. MLO-Y4 cells were incubated for 25 minutes in serum-free medium in the presence or absence of 50 μM PD98059. Subsequently, 17β-estradiol, 17α-estradiol, 17β-estradiol-BSA or estratriene-3-ol (10⁻⁸ M) were added and cells incubated for an additional 5 min. Cell lysates were prepared and proteins were separated by electrophoresis in polyacrylamide gels and transferred to PVDF membranes. Western blotting was performed using a specific antibody recognizing phosphorylated ERKs 1 and 2, followed by reblotting with an antibody recognizing total ERKs. Blots were developed by enhanced chemiluminescence.

FIG. 10 is a series of bar chart graphs which demonstrate that the specific inhibitor of ERK activation, PD98059, abolishes the anti-apoptotic effect of 17β-estradiol and related compounds. MLO-Y4 osteocytic cells were pretreated for 1 hour with 50 μM PD98059 before the addition of 10⁻⁸ M 17β-estradiol, 17α-estradiol, or 17β-estradiol-BSA. Apoptosis was induced by incubation with the pro-apoptotic agent dexamethasone for 6 hour and quantified as described in FIG. 3. * indicates p<0.05 versus the corresponding control group without dexamethasone, by ANOVA (Student-Newman-Keuls method).

FIG. 11 illustrates that unlike 17β estradiol, estratriene-3-ol does not transactivate an estrogen response element through ERα. The human ERα was overexpressed in 293 cells lacking ERα along with a reporter construct containing 3 copies of an estrogen response element driving the luciferase gene. Light units were counted and normalized to coexpressed b-galactosidase activity to control for differences in transfection efficiency. Results represent percent stimulation compared to ERα transfected cells, but not treated with the two agents. Each bar represents mean of duplicate experiments +/−SEM. *p<0.001 vs. cells not exposed to the sterols.

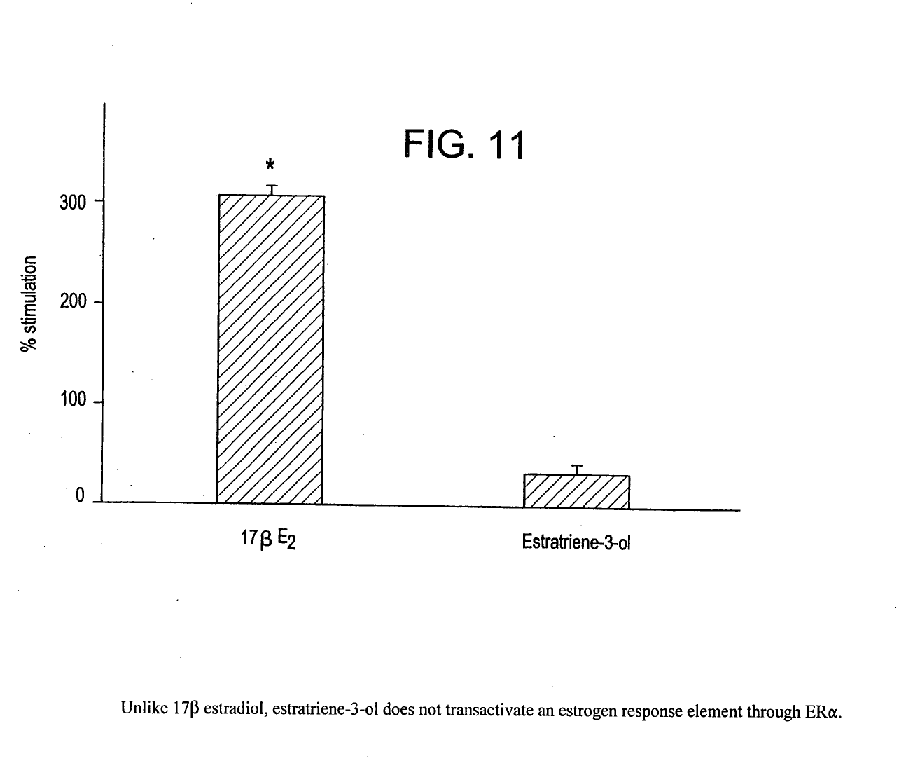

FIG. 12 is an illustration of the chemical structures of certain 3-ring compounds: [2S-(2a,4aα,10aβ)]-1,2,3,4,4a,9,10,10a-octahydro-7-hydroxy-2-methyl-2-phenanthrenemethanol (PAM) and [2S-(2a,4aα,10aβ)]-1,2,3,4,4a,9,10,10a-octahydro-7-hydroxy-2-methyl-2-phenanthrenecarboxaldehyde (PACA).

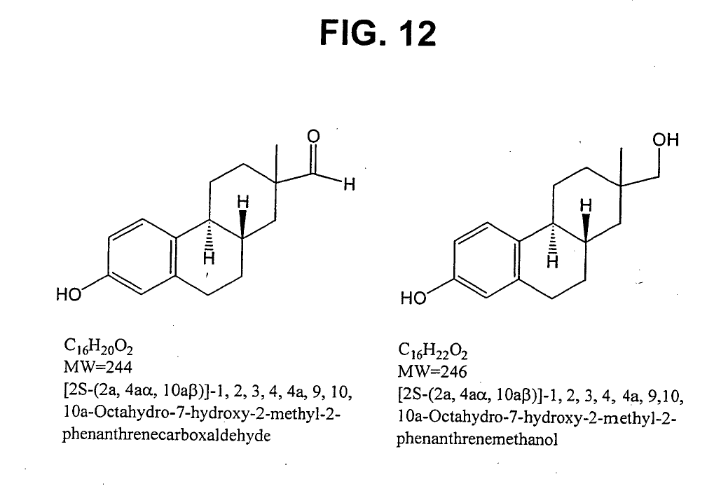

FIG. 13 illustrates the generalized core ring structures with numbered carbons (FIG. 13a) 4-ring structure, (FIG. 13b) 3-ring structure, (FIG. 13c) 2-ring structure (fused), and (FIG. 13d) 2-ring structure (non-fused).

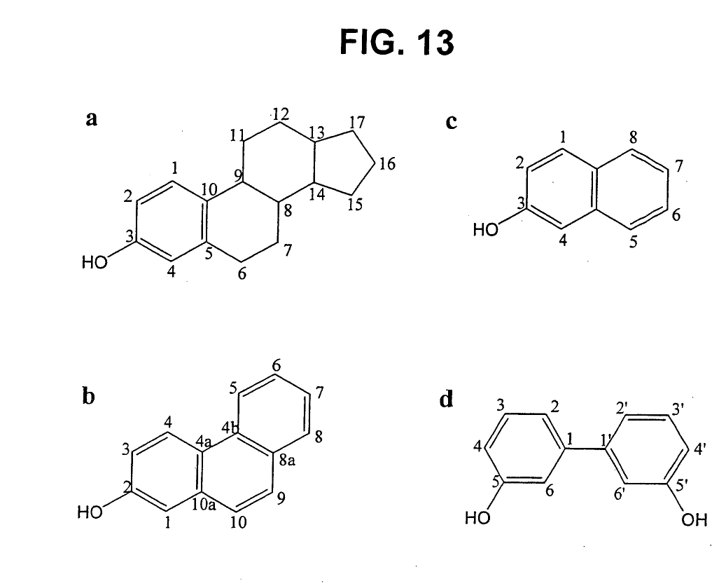

FIG. 14 is an illustration of three mechanisms of estrogen activity: FIG. 14A (anti-apoptotic effect of estrogen), FIG. 14B (anti-remodeling effect of estrogen) and FIG. 14C (feminizing effect of estrogen).

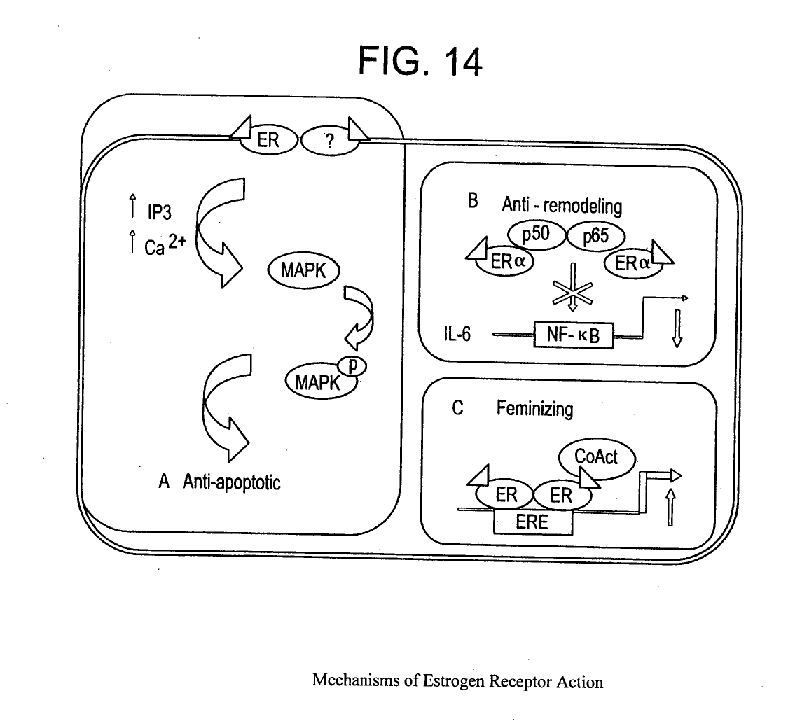

FIG. 15 compares the activity of the anti-resorptive (e.g., 17β-estradiol) versus non-anti-resorptive agents [e.g., estratriene-3-ol or intermittent PTH] on osteoblast and osteocyte apoptosis. Bone formation occurs only on sites of previous osteoclastic bone resorption, i.e., on sites undergoing remodeling. Each remodeling cycle is a transaction that, once consummated, is irrevocable. As shown in the right panel, agents with anti-apoptotic properties that do not have anti-resorptive/anti-remodeling properties rebuild more bone and therefore, increase the overall bone mass because they will not decrease the number of the remodeling units (i.e., the number of transactions). In addition, by decreasing the prevalence of osteoblast apoptosis, the active compounds expand the pool of mature osteoblasts at sites of new bone formation and allow these cells more time to make bone. Moreover, by upholding the osteocyte-canalicular network by preventing osteocyte apoptosis, both classical antiresorptive agents like 17β-estradiol and agents that are not anti-resorptives are expected to have anti-fracture efficacy over and above that resulting from their effects on bone mass.

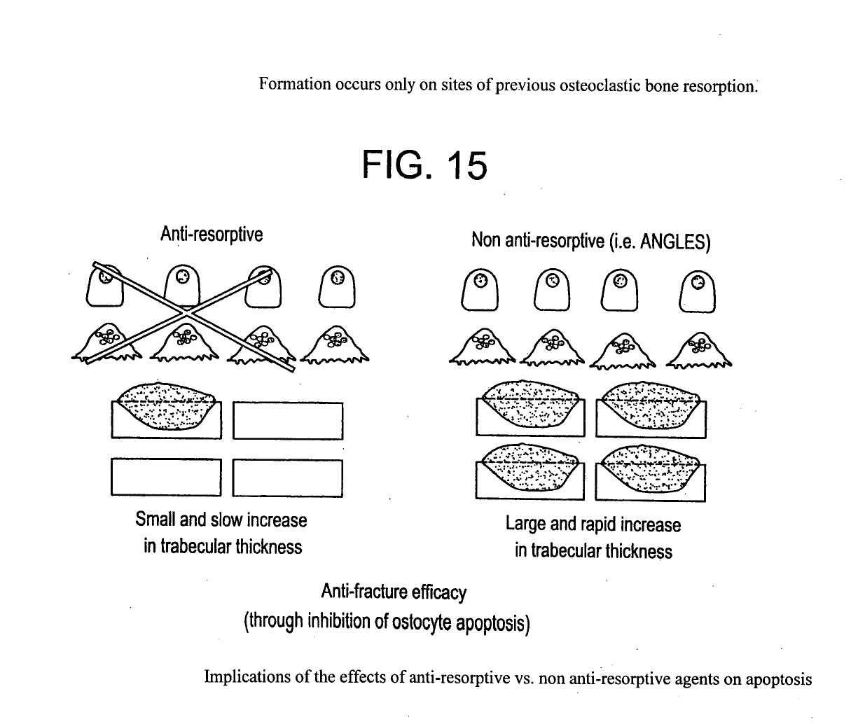

FIG. 16A is a table of examples of R1 and R2 substitutions on the compound illustrated in FIG. 1.

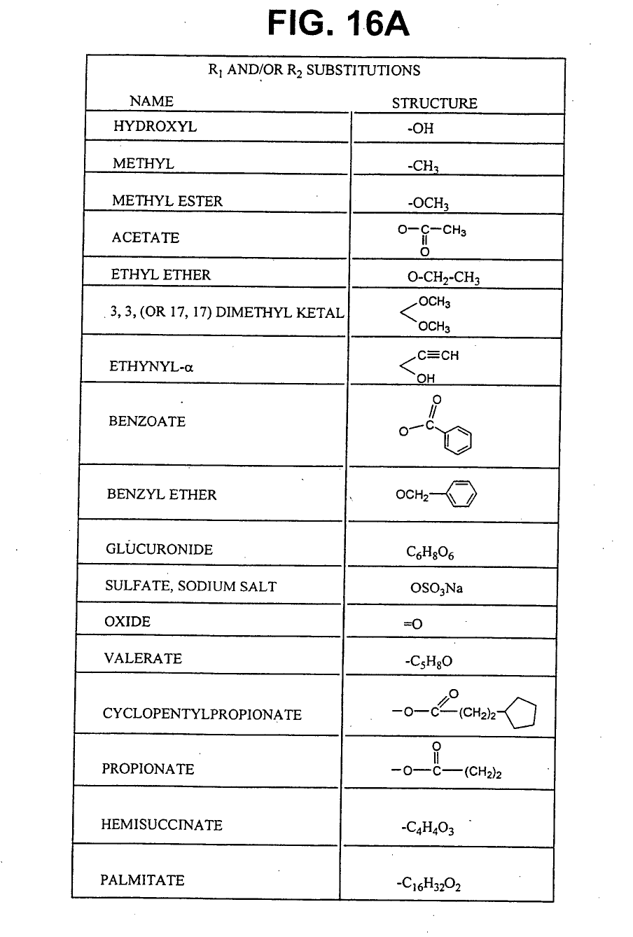

FIG. 16B provides the molecular structures of α and β estradiol.

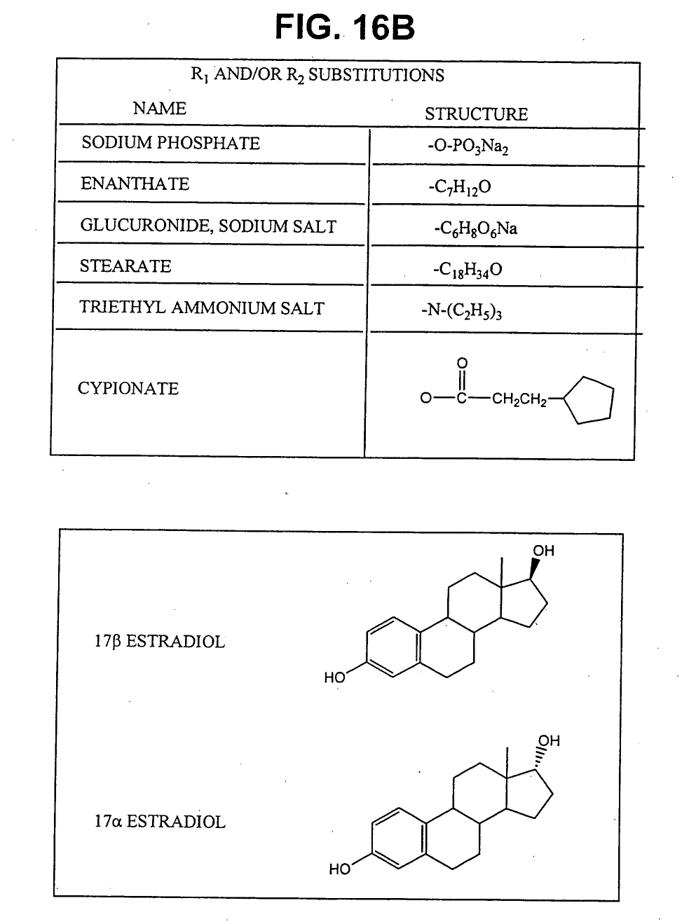

FIG. 17 provides the chemical structures of estratrienes with anti-apoptotic properties.

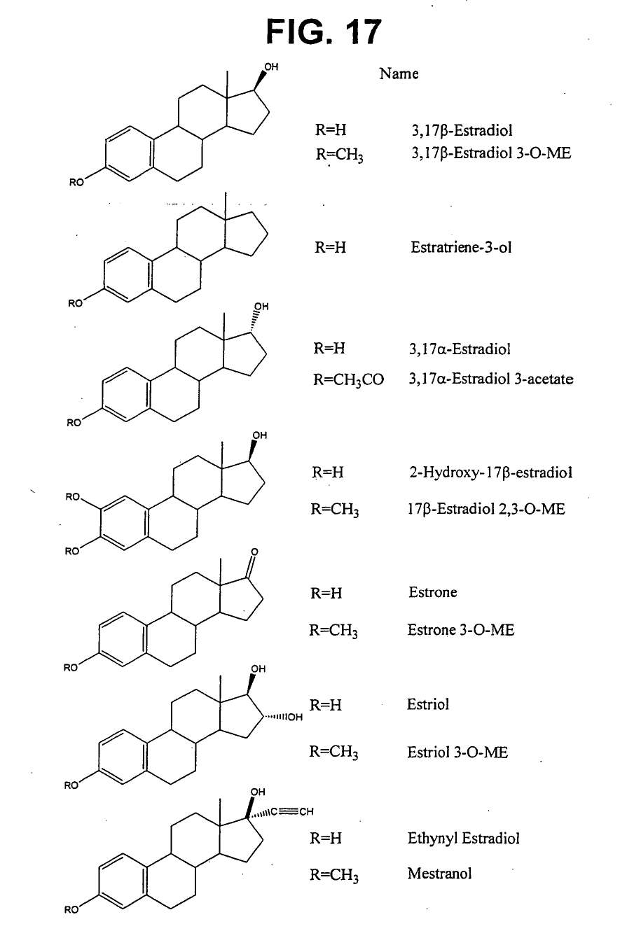

FIG. 18 provides the chemical structures of estradiol, phenol and diphenols with anti-apoptotic properties.

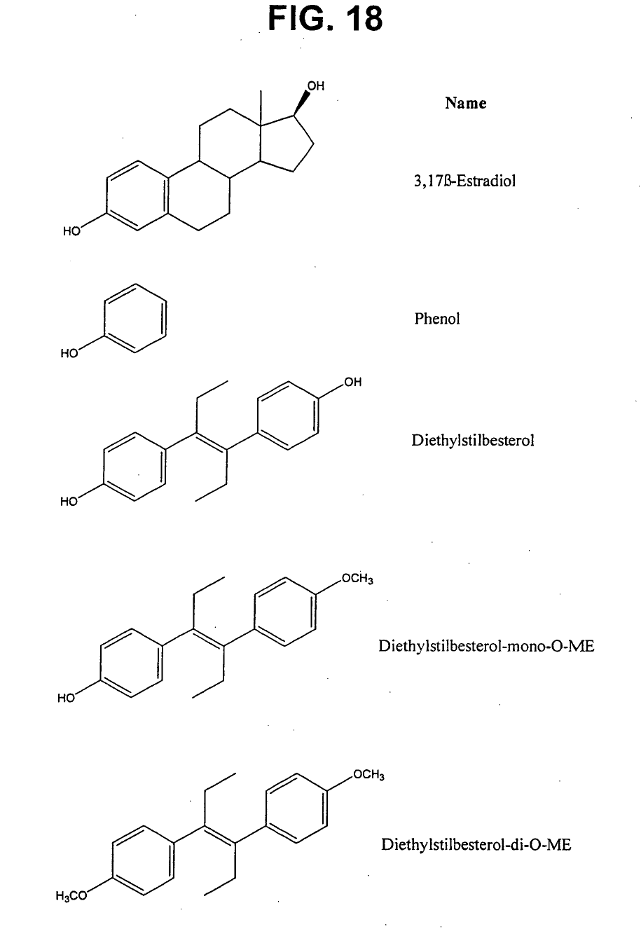

FIG. 19 depicts the effect of 17β estradiol on the transcriptional activity of a minimal ERE containing gene promoter and the blockade of this effect by a peptide (αII) recognizing the ligand-induced specific conformational change of the estrogen receptor protein. 293, human kidney cells, were transiently transfected with a plasmid carrying the ER-specific αII peptide with the GAL4-DNA binding domain inserted upstream of the peptide sequence, an ERE/IL-6 promoter-driven luciferase reporter plasmid and a β-galactosidase (β-gal)-containing plasmid. The ERE-luciferase construct carried three copies of the Xenopus vitellogenin ERE driving the luciferase gene in the pGL3-Basic vector (Promega). * indicates p<0.05 versus cells transfected with the peptide αII, by ANOVA (Student-Newman-Keuls method).

FIG. 20 depicts the effect of 17β estradiol on the transcriptional activity of the IL-6 promoter and the blockade of this effect by a peptide (αII) recognizing the ligand-induced specific conformational change of the estrogen receptor protein. The IL-6-luciferase plasmid carried 225 bp of the proximal IL-6 promoter cloned upstream of the luciferase gene in pGL3-Basic. The αII peptide inhibited the transcriptional effects of estrogen on the ERE-dependent transcription model. αII was also shown to block transcription when mediated via protein/protein interaction between the ER and another transcription factor on the IL-6 gene model. * indicates p<0.05 versus cells transfected with the peptide α II, by ANOVA (Student-Newman-Keuls method).

FIG. 21 demonstrates the anti-apoptotic effect of 17β estradiol conjugated with BSA and the lack of inhibition of this particular effect by the conformation sensitive peptide αII. The effect of the peptide on apoptosis was assayed using etoposide as the apoptotic stimulus. Upon etoposide treatment, cells that had been transfected with the ER and treated with 17β-BSA were protected from apoptosis. Following co-transfection of the GAL4-driven peptide, cells remained resistant to etoposide-induced apoptosis indicating that the peptide did not inhibit the protective, anti-apoptotic action of the ER (FIG. 21). * indicates p<0.05 versus cells transfected with the peptide αII, by ANOVA (Student-Newman-Keuls method).

### DETAILED DESCRIPTION OF THE INVENTION

The invention as disclosed provides a method to increase bone mass without compromising bone quality, through the administration to a host of an effective amount of a compound that binds to the estrogen or androgen receptor so as to trigger the anti-apoptotic signalling pathway, but with minimal or no resultant transcriptional activity.

In an optimal embodiment using this invention, an anabolic effect will be established by demonstrating increased bone formation, assessed by double tetracycline labeling (Weinstein R. S. In Disorders of Bone and Mineral Metabolism (eds. Coe and Favus) Raven Press, 1992, pp. 455-474) and a continuous increase in BMD, assessed by DEXA (Jilka et al. J. Clin. Invest. 97:1732-1740, 1996) for at least five years, along with increased, or at least no decreased quality or strength.

This invention is based on the fundamental discovery that bone loss occurs because of an increase in osteoblast apoptosis, which can be inhibited by a compound that binds to an estrogen or androgen receptor (which induces the phosphorylation of ERKs) with minimal or no resultant transcriptional activity. The discovery of this fundamental pathway allows the selection of compounds which provide a maximum effect on bone mass and strength.

Therefore, in a first embodiment, a method for increasing bone mass in a host at least 10% without a loss in bone quality or strength is provided that includes administering an effective amount of a compound that (i) binds to the estrogen α or β receptor (or the equivalent receptor in the host animal) with an association constant of at least 10′ M⁻¹ , and preferably, at least 10¹⁰ M⁻¹ ; (ii) (a) induces estrogenic gene transcriptional activity at a level that is no greater than 10% that of 17β-estradiol, and preferably no greater than 5, 1 or even 0.1% that of 17β-estradiol when administered in vivo at a dosage of at least 0.1 ng/kg body weight or in vitro in cells with natural estrogen receptors or transfected with estrogen receptors or (b) induces an increase in uterine weight of no more than 10% that of estrogen (or the equivalent compound in a host animal); (iii) induces the phosphorylation of extracellular signal regulated kinase (ERK) when administered in vivo at a dosage of at least 0.1 ng/kg body weight or at a concentration of 10⁻¹¹ to 10⁻⁷ M in vitro in cells with natural estrogen receptors or transfected with estrogen receptors and (iv) has an anti-apoptotic effect on osteoblasts and osteocytes at an in vivo dosage of at least 0.1 ng/kg body weight or at a concentration of 10⁻¹¹ to 10⁻⁷ M in vitro in cells with natural estrogen receptors or transfected with estrogen receptors. In another aspect of this first embodiment of this invention, the compound is not an estrogen compound, as that term is defined herein. In another aspect of this first embodiment, the compound is an estrogen compound which is converted to a nonestrogen by attaching a substituent which prevents the compound from entering the cell, but which does not significantly affect the binding of the compound to the estrogen cell-surface estrogen receptor.

In a second embodiment, a method for increasing bone mass in a host at least 10% per year without a loss in bone strength or quality is provided that includes administering an effective amount of a compound that (i) binds to the androgen receptor (or the equivalent receptor in the host animal) with an association constant of at least 10⁸ M⁻¹, and preferably, at least 10¹⁰ M⁻¹: (ii) (a) induces androgenic gene transcriptional activity at a level that is no greater than 10% that of testosterone, and preferably no greater than 5, 1 or even 0.1% that of testosterone when administered in vivo at a dosage of at least 0.1 ng/kg body weight or in vitro in cells with the natural androgen receptor or transfected with the androgen receptor or (b) induces an increase in muscle weight of no more than 10% that which is induced by testosterone (or the equivalent compound in a host animal); (iii) induces the phosphorylation of extracellular signal regulated kinase (ERK) when administered in vivo at a dosage of at least 0.1 ng/kg body weight, or at a concentration of 10⁻¹¹ to 10⁻⁷ M in vitro in cells with the natural androgen receptor or transfected with the androgen receptor; and (iv) has an anti-apoptotic effect on osteoblasts and osteocytes at an in vivo dosage of at least 0.1 ng/kg body weight or at a concentration of 10⁻¹¹ to 10⁻⁷ M in vitro in cells with the natural androgen receptor or transfected with the androgen receptor. In another aspect of the second embodiment, the compound is not an androgen. In another aspect of this second embodiment, the compound is an androgen compound which is converted to a nonandrogen by attaching a substituent which prevents the compound from entering the cell containing the cell-surface androgen receptor.

In other aspects of the first or second embodiment of this invention, the compound also has a pro-apoptotic effect on osteoclasts at an in vivo dosage of at least 0.1 ng/kg body weight or in vitro in cells with the natural androgen receptor or transfected with the androgen receptor.

Therefore, in a third embodiment, a method for selecting a compound that increases bone mass in a host at least 10% without a loss in bone strength or quality is provided that includes evaluating whether the compound (i) binds to the estrogen or androgen receptor (or the equivalent receptor in the host animal) with an association constant of at least 10⁸ M⁻¹ , and preferably, at least 10¹⁰ M⁻¹ : (ii) (a) induces estrogenic or androgenic gene transcriptional activity at a level that is no greater than 10% that of testosterone or 17β-estradiol, and preferably no greater than 5, 1 or even 0.1% that of 17β-estradiol or testosterone, as appropriate, when administered in vivo at a dosage of at least 0.1 ng/kg body weight or at a concentration of 10⁻¹¹ to 10⁻⁷ M or in vitro in cells with the natural androgen or estrogen receptor or transfected with the androgen or estrogen receptor or (b) induces an increase in uterine or muscle weight, as appropriate, of no more than 10% that which is induced by 17β-estradiol or testosterone (or the equivalent compound in a host animal); (iii) induces the phosphorylation of extracellular signal regulated kinase (ERK) when administered in vivo at a dosage of at least 0.1 ng/kg body weight or at a concentration of 10⁻¹¹ to 10⁻⁷ M in vitro in cells with the natural androgen or estrogen receptor or transfected with the androgen or estrogen receptor; and (iv) has an anti-apoptotic effect on osteoblasts at an in vivo dosage of at least 0.1 ng/kg body weight or in vitro in cells with the natural androgen or estrogen receptor or transfected with the androgen or estrogen receptor.

Compounds selected according to the criteria provided herein can also be used as for the augmentation of bone mass and/or fracture prevention in diseases characterized by low bone mass and increased fragility. The compounds can be used to treat bone disease states in which osteoblastogenesis is decreased, such as senile osteoporosis, and glucocorticoid-induced osteoporosis—especially in growing children and adolescents, in whom interfering with bone remodeling is detrimental.

I. Definitions

An estrogen compound, as used herein, refers to a four ring steroidal compound which possesses the biological activity of an estrus-producing hormone, or its conjugated and esterified derivative, or a derivative thereof of same chemical composition and structure but which does not possess the biological activity of the active form because it exhibits a different stereochemistry from the active form. Nonlimiting examples of estrogens include broparestrol, chlorotrianisene, dienoestrol, epimestrol, equilin, estrapronicate, estropipate, ethinylestradiol, fosfestrol, hydroxyesetrone, mestranol, estradiol, estriol, conjugated and esterified estrogens, estrone, polyestradiol, promestriene, quinestradol, quinestrol, stilbestrol, and zeranol.

An androgen compound, as used herein, refers to a four ring steroidal compound which can be produced in the testis or adrenal cortex, or is a synthetic hormone, which acts to regulate masculine secondary sexual characteristics, or a derivative thereof of same chemical composition and structure but which does not possess the biological activity of the active form because it exhibits a different stereochemistry from the active form. Nonlimiting examples include boldenone, clostebol, danazol, drosstanolone, epitiostanol, ethylestrenol, fluoxymesterone, formebolone, furazabol, mepitiostane, mesterolone, methandienone, methenolone, methyltestosterone, nandrolone, norethandrolone, oxabolone, oxymetholone, prasterone, quinbolone, staolone, stanozolol, testosterone, and trenbolone.

As known, estrogens and androgens have chiral carbons, and thus can exist in a number of stereochemical configurations. Typically, for example, the 17βhydroxy estrogens have biological activity while the 17α hydroxy estrogens have very little effect on sexual characteristics (and induce little hormone-like gene transcriptional activation). For the purpose of this specification, any stereochemical configuration, including either the biologically active or the biologically inactive or less active structure, can be used, as long as the compound satisfies the specifically itemized criteria of the invention.

The catalogue entitled “Steroids” from Steraloids Inc., Wilton, N.H., provides a list of over 3000 steroids, with numerous estrogen and androgen derivatives. The catalog can be obtained by contacting the company and is also currently available on the internet at http://www.steraloids.com. One can select and purchase compounds from this library, which are all commercially available and thus easy to obtain and evaluate, for use in this invention. One can also use known estrogen and androgen receptor binding compounds.

The term “bone mass” refers to the mass of bone mineral and is typically determined by Dual-Energy X-Ray Absorbtiometry (DEXA).

The term “bone strength” refers to resistance to mechanical forces and can be measured by any known method, including vertebrae compression strength or three point-bending of long bones.

The term “bone quality” refers to normal collagen orientation without excessive accumulation of unmineralized bone matrix, and can be measured by any known method, including undecalcified bone histomorphometry.

The term “bone anti-resorption agent” refers to a compound that blocks bone resorption by suppressing remodeling or the activity and/or lifespan of osteoclasts.

The term “osteopenia” refers to decreased bone mass below a threshold which compromises structural integrity.

As used herein, the terms “metabolic bone disease”, “orthopedic bone disease” or “dental disease” are defined as conditions characterized by decreased bone mass and/or structural deterioration of the skeleton and/or teeth.

As used herein, the term “apoptosis” refers to programmed cell death characterized by nuclear fragmentation and cell shrinkage as detected by morphological criteria and Terminal Uridine Deoxynucleotidal Transferase Nick End Labeling (TUNEL) staining.

The term “host”, as used herein, refers to any bone-containing animal, including, but not limited to humans, other mammals, canines, equines, felines, bovines (including chickens, turkeys, and other meat producing birds), cows, and bulls.

II. Compounds Useful in the Invention

A. Estrogen Compounds that Bind to the Estrogen α or β Receptor with an Association Constant of at Least 10⁸ M⁻¹, and Preferably, at Least 10¹⁰ M⁻¹, but which Exhibit Little Transcriptional Activation

According to the present invention, one can easily select estrogen compounds that significantly increase bone mass by evaluating them according to the disclosed criteria.

1. Binding to the Estrogen α or β Receptor

A compound should be selected that binds to the estrogen α or β receptor (or the equivalent receptor in the host animal) with an association constant of at least 10⁸ M⁻¹, and preferably, at least 10¹⁰ M⁻¹. This constant can be measured by any known technique, including receptor binding assays whereby ligand binding affinities are determined by competitive radiometric binding assays using 10 nM [³H] estradiol as tracer, purified estrogen receptor preparations, or cell cytosol preparations, or intact cells, during one hour incubation at room temperature or overnight at 4°. Bound receptor-ligand complex is absorbed using hydroxylapatite.

The estrogen α and β receptor subtypes have significantly different primary sequences in their ligand binding and transactivation domains. ERα and ERβ show a 56% amino acid homology in the hormone binding domain/activation function-1 region, and only 20% homology in their A/B domain/activation function-1 region. The difference between ERα and ERβ structure suggests that some compounds might bind ERβ or ERβ, but not both. All such selectively binding compounds are considered to fall within the scope of this invention.

Estrogen compounds include those described in the 11th Edition of “Steroids” from Steraloids Inc., Wilton, N.H., which bind to the estrogen receptor with an association constant of at least 10⁸ M⁻¹ , and preferably, at least 10¹⁰ M⁻¹ .

2. Minimal Effect on Estrogen-Induced Transcriptional Activation

In this embodiment, an estrogen compound is selected that has a minimal effect on estrogen-induced transcriptional activation (or suppression). The basis for this requirement is that it has been discovered that apoptosis of osteoblasts is decreased by receptor binding, in the absence of transcriptional activation by estrogen-type compounds. Therefore, to provide a maximum therapeutic efficacy on bone without causing unrelated and undesired side estrogen-related effects, estrogen receptor ligands with minimal transcriptional effects should be used.

To accomplish this separation of receptor binding and transcriptional activity, a compound should be selected that induces estrogenic gene transcriptional activity at a level that is no greater than 10% that of 17β-estradiol, and preferably no greater than 5, 1 or even 0.1% that of 17β-estradiol when administered in vivo at a dosage of at least 0.1 ng/kg body weight or in vitro in cells with natural estrogen receptors or transfected with estrogen receptors or which induces an increase in uterine weight of no more than 10% that of estrogen (or the equivalent compound in a host animal).

One can determine whether a selected compound induces estrogenic transcriptional activity at a level that is no greater than 10% that of 17β-estradiol, and preferably no greater than 5, 1 or even 0.1% that of 17β-estradiol when administered in vivo at a dosage of at least 0.1 ng/kg body weight, by administering the selected compound to a host, and then monitoring the level of induction or suppression of a surrogate marker of estrogenic transcriptional activity. Nonlimiting examples of surrogate markers of estrogenic transcriptional activation, include, but are not limited to, the expression of the complement C-3 gene and lactoferin in the uterus.

In an alternative embodiment, the level of estrogen-induced transcriptional activity can be assessed in vitro. One can determine whether a selected compound induces transcriptional activity at a level that is no greater than 10% that of 17β-estradiol, and preferably no greater than 5, 1 or even 0.1% that of 17β-estradiol in vitro using cells with natural estrogen receptors or transfected with estrogen receptors, by monitoring the level of induction or suppression of a surrogate marker. Nonlimiting examples of genes induced or repressed by estrogen include, but are not limited to, complement C-3, lactoferin, or interleukin-6. A preferred marker gene for estrogenic transcriptional activity is a minimal gene containing one or more copies of the ERE driving a reporter gene such as luciferase.

Examples of cell lines that can be used include human uterine HeLa cells, human embryonic kidney cells 293, murine osteocytic MLO-Y4 cells and murine osteoblastic calvaria derived cells.

One can assess the increase in uterine weight after administration of the selected compound in vivo. Preferred compounds induce an increase in uterine weight of no more than approximately 10% that of estrogen (or the equivalent compound in a host animal). This can be easily tested according to known protocols. For example, in experimental mice, uteri are removed and cleaned of adjacent ligaments and fat. Wet weight is determined on a Mettler PB303 microgram balance (Toledo) and compared to total body weight (mg/100 g BW) as an index of the estrogenic status of the animals. In women, similar assessment can be performed by uterine ultrasound.

Examples of estrogen compounds that do not induce significant estrogen-like transcriptional activity include, but are not limited to estratriene-3-ol, 17α-estradiol, 17β-estradiol conjugated with BSA.

3. Induction of the Phosphorylation of Extracellular Signal Regulated Kinase (ERK)

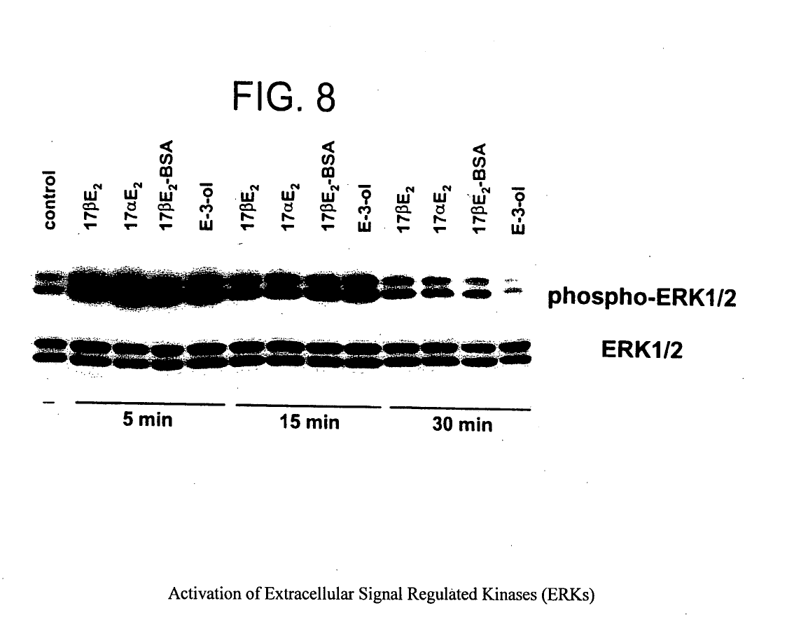

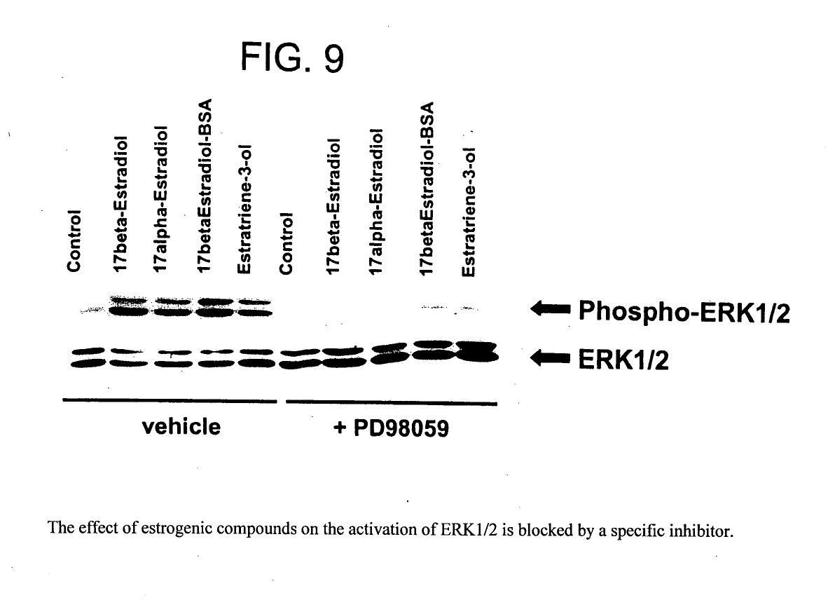

The selected compound should induce the phosphorylation of ERKs at a concentration of 10⁻¹¹ to 10⁻⁷ M in vitro in cells with natural estrogen receptors or transfected with estrogen receptors using any known method, including but not limited to, the method set out in FIGS. 8 and 9 and Examples 7-9.

The phosphorylation of ERKs is easily assessed in vitro using osteoblastic or osteocytic cells with natural estrogen receptors or cells transfected with estrogen receptors. Examples of the evaluation of the phosphorylation of ERK in MLO-Y4 cells are provided in FIGS. 8 and 9 and Examples 7-9. Other appropriate cell models include osteoblastic cells isolated from neonatal murine calvaria.

4. Anti-Apoptotic Effect on Osteoblasts at an In Vivo Dosage of at Least 0.1 ng/kg Body Weight or at an In Vitro Concentration of 10⁻¹¹ to 10⁻⁷ m or Less.

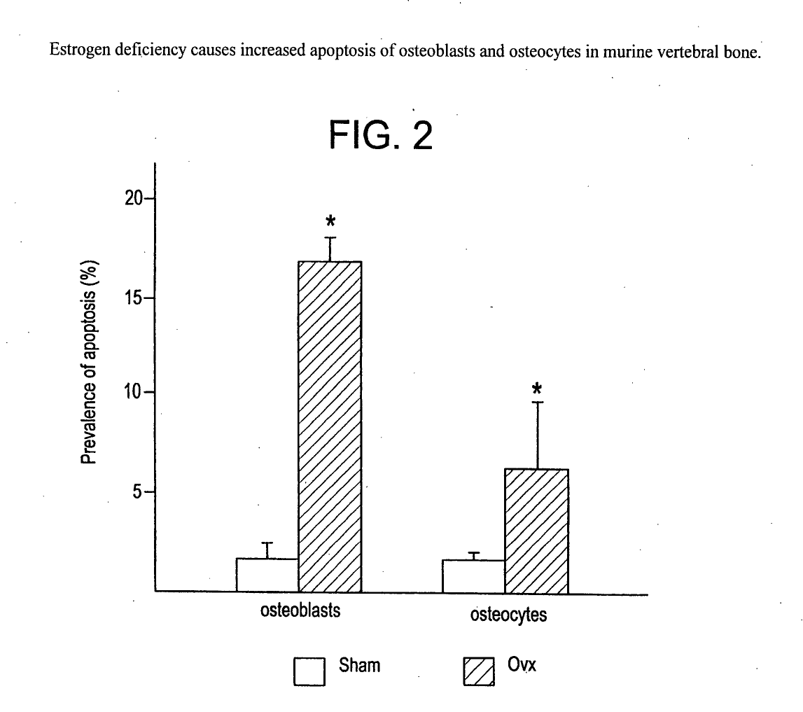

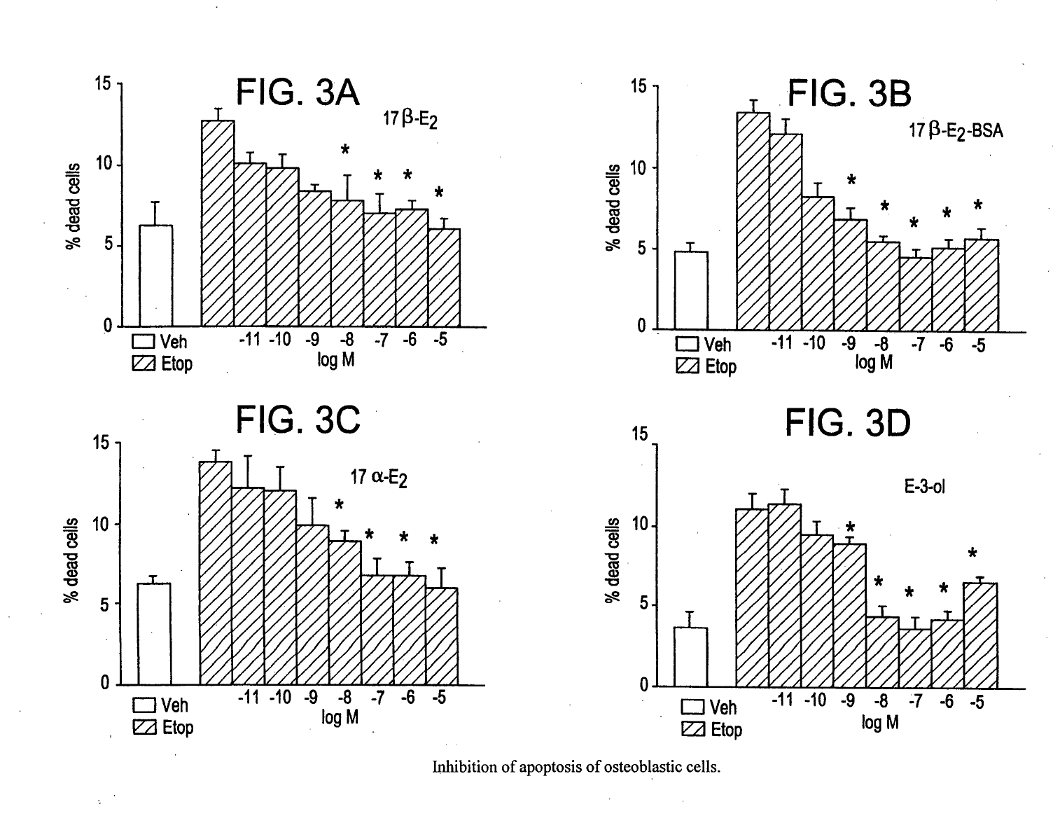

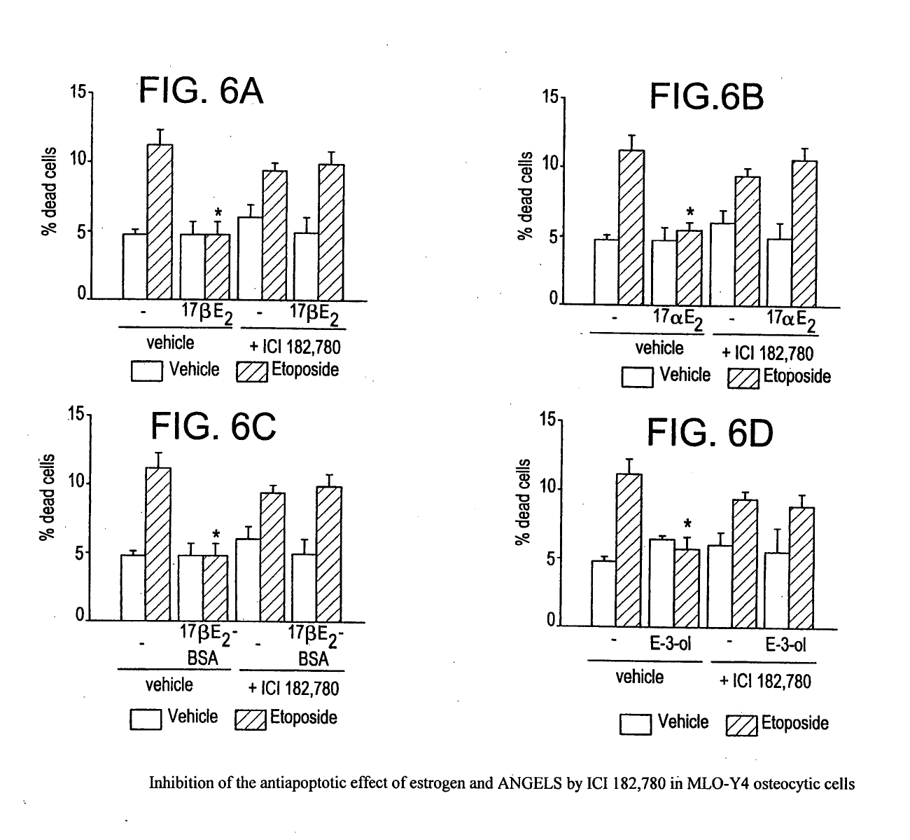

The anti-apoptotic effect on osteoblasts in vivo can be assessed by any known method, including by the method described in FIG. 2 and Example 3. The anti-apoptotic effect in vitro can be assessed by any known method including the methods described in FIGS. 3-7 and 10, and Examples 2-6 and 9.

B. Nonestrogen Compounds that Bind to the Estrogen α or β Receptor with an Association Constant of at Least 10⁸ M⁻¹ and Preferably, at Least 10¹⁰ M⁻¹, but which Exhibit Little Transcriptional Activation

1. Nonestrogen Compound which Binds to the Estrogen α or Receptor

A nonestrogen compound, as used herein, refers to a compound other than an estrogen, as that term is defined above, which binds to the estrogen α or β receptor with an association constant of at least 10⁸ M⁻¹ and preferably, at least 10¹⁰ M⁻¹ . There are a number of reported compounds which are not estrogens but which bind to the estrogen receptor.

Examples include the aryl-substituted pyrazole described by Sun et al., Novel Ligands that Function as Selective Estrogens or Antiestrogens for Estrogen Receptor-α or Estrogen Receptor-β, Endocrinology, Volume 140, No. 2 (1999), one example of which is illustrated below.

In an alternative embodiment, an estrogen or nonestrogen compound is covalently linked to a second moiety that does not significantly interfere with the binding to the estrogen receptor but which does substantially prevent the estrogen from entering the cell. In one example, the second moiety is a protein such as bovine serum albumin, polyethlene glycol or dextran or liposomes. In another embodiment, the second moiety is not a protein or peptide, but for polar, steric, or other reasons, prevents cell penetration. Examples of these types of moieties include carboxylate, ammonium, and sulfide. A “linking moiety” as used herein, is any divalent group that links two chemical residues, including but not limited to alkyl, alkenyl, alkynyl, aryl, polyalkyleneoxy (for example, —[(CH₂)ₙO—]ₙ—), —C₁₋₆alkoxy—C₁₋₁₀alkyl—, —C₁₋₆alkylthio—C₁₋₁₀ alkyl-, —NR₃—, and —(CHOH)ₙCH₂OH, wherein n is independently 0, 1, 2, 3, 4, 5, or 6, which can be attached at either end of the linking moiety to the structures of interest by any suitable functional groups. In an alternative embodiment, the linking moiety can be a bifunctional linker moiety of the formula X—(CH₂)ₙ—Y, wherein X and Y are functional groups capable of linking, including those independently selected from the group consisting of hydroxyl, sulfhydryl, carboxyl and amine groups, and n can be any integer between one and twenty four.

C. Androgen Compounds that Bind to the Androgen Receptor with an Association Constant of at Least 10⁸ M⁻¹, and Preferably, at Least 10¹⁰ M⁻¹, But Which Exhibit Little Transcriptional Activation

According to the present invention, one can also easily select androgenic compounds that significantly increase bone mass by evaluating them according to the disclosed criteria.

1. Binding to the Androgen Receptor

A compound should be selected that binds to the androgen receptor (or the equivalent receptor in the host animal) with an association constant of at least 10⁸ ⁻¹ , and preferably, at least 10¹⁰ M⁻¹ . The androgen receptor binding association constant is defined as the concentration of the ligand capable of saturating 50% of the unoccupied receptors. This constant can be measured by any known technique, including receptor binding assays whereby ligand binding affinities are determined by competitive radiometric binding assays using 10 nM [³H] of the synthetic androgen RU1881 as tracer, purified androgen receptor preparations, or cell cytosol preparations, or intact cells, during one hour incubation at room temperature or overnight at 4° C. Bound receptor-ligand complex is absorbed using hydroxylapatite. Androgen compounds include those described in the 11th Edition of “Steroids” from Steraloids Inc., Wilton, N.H., which bind to the androgen receptor with an association constant of at least 10′ M⁻¹ , and preferably, at least 10¹⁰ M⁻¹ . 2. Minimal Effect on Androgen-Induced Transcriptional Activation

In this embodiment, an androgen compound is selected that has a minimal effect on androgen-induced transcriptional activation. The basis for this requirement, is that it has been discovered that apoptosis of osteoblasts is decreased by receptor binding in the absence of transcriptional activation by androgen-type compounds. Therefore, to provide a maximum therapeutic efficacy on bone without causing unrelated and undesired androgen-related effects, androgen receptor ligands with minimal transcriptional activity should be used.

To accomplish this separation of receptor binding and transcriptional activity, a compound should be selected that induces androgenic transcriptional activity at a level that is no greater than 10% that of testosterone, and preferably no greater than 5, 1 or even 0.1% that of testosterone when administered in vivo at a dosage of at least 0.1 ng/kg body weight or in vitro in cells with natural androgen receptors or transfected with androgen receptors or induces an increase in prostate specific antigen (PSA) prostatic serum androgen of no more than 10% that of testosterone (or the equivalent compound in a host animal).

One can determine whether a selected compound induces androgenic gene transcriptional activity at a level that is no greater than 10% that of testosterone, and preferably no greater than 5, 1 or even 0.1% that of testosterone when administered in vivo at a dosage of at least 0.1 ng/kg body weight, by administering the selected compound to a host, and then monitoring the level of induction or suppression of a surrogate marker of androgenic transcriptional activity. Nonlimiting examples of surrogate markers of androgenic transcriptional activation, include, but are not limited to prostate specific antigen (PSA).

In an alternative embodiment, the level of androgen-induced transcriptional activity can be assessed in vitro in osteoblastic or osteocytic cells with natural androgen receptors or traf calvaria cells, MLO-Y4 osteocytic cells and HeLa cells.

Alternatively, one can assess the increase in PSA serum levels after administration of the selected compound. Appropriate compounds induce an increase in PSA cells transfected with androgen receptors. Examples of such cell types include, primary cultures of PSA of no more than approximately 10% that of testosterone (or the equivalent compound in a host animal). This can be easily tested according to known protocols.

Examples of androgenic compounds that do not induce significant androgenic-like transcriptional activity include, but are not limited to, testosterone 17β-hemisuccinate conjugated with BSA.

3. Induction of the Phosphorylation of Extracellular Signal Regulated Kinase (ERK)

The selected compound should induce the phosphorylation of ERKs when administered in vivo at a dosage of at least 0.1 ng/kg body weight or at a concentration of 10⁻¹¹ to 10⁻⁷ M in vitro in cells with natural androgenic receptors or transfected with androgenic receptors.

The phosphorylation of ERK in a host can be assessed in biopsies, for example from bone, using immunohistostaining with specific antibodies against phosphorylated ERKs. Alternatively, the phosphorylation of ERK is also easily assessed in vitro using osteoblastic or osteocytic cells with natural androgen receptors or cells transfected with androgen receptors. Examples of the evaluation of the phosphorylation of ERK in MLO-Y4 cells are provided FIGS. 8 and 9 and Examples 7-9.

4. Anti-Apoptotic Effect on Osteoblasts and Osteocytes at an In Vivo Dosage of at Least 0.1 ng/kg Body Weight or at an In Vitro Concentration of 10⁻¹¹ to 10⁻⁷ m or Less.

The anti-apoptotic effect on osteoblasts and osteocytes can be assessed in vivo by any known method, including the method described in FIG. 2 and Example 1; and in vitro by any known method, including the method described in FIGS. 3-7 and 10 and Examples 2-6 and 9.

D. Nonandrogen Compounds that Bind to the Androgen Receptor with an Association Constant of at Least 10⁸ M⁻¹, and Preferably, at Least 10¹⁰ M⁻¹, but which Exhibit Little Transcriptional Activation

A nonandrogenic compound, as used herein, refers to a compound other than an androgen, as that term is defined above, which binds to the androgenic receptor with an association constant of at least 10⁸ M⁻¹ and preferably, at least 10¹⁰ M⁻¹ . There are a number of reported compounds which are not androgens but which bind to the androgen receptor. Examples include testosterone 17β-hemisuccinate conjugated with BSA.

In an alternative embodiment, an androgen compound is covalently linked to a second moiety that does not significantly interfere with the binding to the androgen receptor but which does substantially prevent the androgen from entering the cell. In one example, the second moiety is a protein such as bovine serum albumin. In another embodiment, the second moiety is not a protein or peptide, but for polar, steric, or other reasons, prevents cell penetration. Examples of these types of moieties include dextran or plyethelene glycol.

E. Other Compounds that can be Used to Increase Bone Mass.

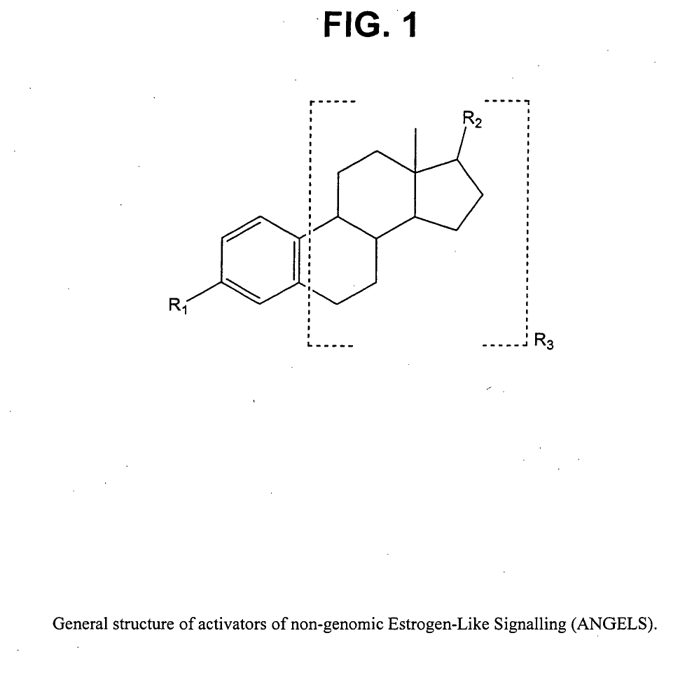

Other nonlimiting examples of compounds that can be used in the present invention to increase bone mass include those having a terminal phenyl ring and at least a second carbon ring. In addition to these required structures, the compound may have a number of R groups attached to any available site on the phenyl ring or elsewhere. These R groups may be selected from inorganic or organic atoms or moieties. Representative R groups are provided, although the invention is not to be limited by these examples:
(a) The R₁ or R₂ groups may include a hydroxyl group or an inorganic R group including any of a halogen, an amide, a sulfate, a nitrate, fluoro, chloro, or bromo groups. Additionally, R₁ or R₂ groups such as sodium, potassium and/or ammonium salts may be attached to the alpha or beta positions to replace hydrogen on any available carbon in the structure. The R₁ or R₂ groups may be organic or may include a mixture of organic molecules and ions. Organic R₁ or R₂ groups may include alkanes, alkenes or alkynes containing up to six carbons in a linear or branched array. For example, additional R₁ or R₂ group substituents may include methyl, ethyl, propyl, butyl, pentyl, hexyl, heptyl, dimethyl, isobutyl, isopentyl, tert-butyl, sec-butyl, isobutyl, methylpentyl, neopentyl, isohexyl, hexenyl, hexadiene, 1,3-hexadiene-5-yne, vinyl, allyl, isopropenyl, ethynyl, ethylidine, vinylidine, isopropylidene, methylene, sulfate, mercapto, methylthio, ethylthio, propylthio, methylsulfinyl, methylsulfonyl, thiohexanyl, thiobenzyl, thiophenol, thicyanato, sulfoethylamide, thionitrosyl, thiophosphoryl, p-toluenesulfonate, amino, imino, cyano, carbamoyl, acetamido, hydroxyamino, nitroso, nitro, cyanato, selecyanato, arccosine, pyridinium, hydrazide, semicarbazone, carboxymethylamide, oxime, hydrazone, sulfurtrimethylammonium, semicarbazone, o-carboxymethyloxime, aldehyde hemiacetate, methylether, ethylether, propylether, butylether, benzylether, methylcarbonate, carboxylate, acetate, chloroacetate, trimethylacetate, cyclopentylpropionate, propionate, phenylpropionate, carboxylic acid methylether, formate, benzoate, butyrate, caprylate, cinnamate, decylate, heptylate, enanthate, glucosiduronate, succinate, hemisuccinate, palmitate, nonanoate, stearate, tosylate, valerate, valproate, decanoate, hexahydrobenzoate, laurate, myristate, phthalate, hydroxyl, ethyleneketal, diethyleneketal, formate, chloroformate, formyl, dichloroacetate, keto, difluoroacetate, ethoxycarbonyl, trichloroformate, hydroxymethylene, epoxy, peroxy, dimethyl ketal, acetonide, cyclohexyl, benzyl, phenyl, diphenyl, benzylidene, and cyclopropyl groups. R₁ or R₂ groups may be attached to any of the constituent rings to form a pyridine, pyrazine, pyrimidine, or v-triazine. Additional R₁ or R₂ group substituents may include any of the six-member or five-member rings itemized in section (b) below.
(b) Any compound having, in addition to the terminal phenyl group, at least one heterocyclic carbon ring (shown as R₃ in FIG. 1), which may be an aromatic or non-aromatic phenolic ring with any of the substitutions described in section (a) above, and further may be, for example, one or more of the following structures: phenanthrene, naphthalene, naphthols, diphenyl, benzene, cyclohexane, 1,2-pyran, 1,4-pyran, 1,2-pyrone, 1,4-pyrone, 1,2-dioxin, 1,3-dioxin (dihydro form), pyridine, pyridazine, pyrimidine, pyrazine, piperazine, s-triazine, as-triazine, v-triazine, 1,2,4-oxazine, 1,3,2-oxazine, 1,3,6-oxazine (pentoxazole), 1,2,6-oxazine, 1,4-oxazine, o-isoxazine, p-isoxazine, 1,2,5-oxathiazine, 1,2,6-oxathiazine, 1,4,2-oxadiazine, 1,3,5,2-oxadiazine, morpholine (tetrahydro-p-isoxazine), any of the six-ringed structures listed above being a terminal group in the compound. Additionally, any of the above carbon ring structure may be linked directly, or via a linkage group, to any further heterocyclic aromatic or non aromatic carbon ring including: furan, thiophene (thiofuran), pyrrole (azole), isopyrrole (isoazole), 3-isopyrrole (isoazole), pyrazole (1,2 diazole), 2-isoimidazole (1,3-isodiazole), 1,2,3-triazole, 1,2,4-triazole, 1,2-dithiazole, 1,2,3-oxathiazole, isoxazole (furo(a) monozole), oxazole (furo(b) monazole), thiazole, isothiazole, 1,2,3-oxathiazole, 1,2,4-oxadiazole, 1,2,5-oxadiazole, 1,3,5-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxathiazole, 1,3-oxathiazole, cyclopentane. These compounds, in turn, may have associated R₁ or R₂ groups selected from section (a) or section (b) above that are substituted on the carbon ring at any of the available sites.
(c) Any compound, including those listed above, that may form a cyclopentanophen(a)anthrene ring compound and which, for example, may be selected from the group consisting of 1,3,5(10),6,8-estrapentaene, 1,3,5(10),6,8,11-estrapentaene, 1,3,5(10),6,8,15-estrapentaene, 1,3,5(10),6-estratetraene, 1,3,5(10),7-estratetraene, 1,3,5(10),8-estratetraene, 1,3,5(10),16-estratetraene, 1,3,5(10),15-estratetraene, 1,3,5(10)-estratriene, 1,3,5(10),15-estratriene.
(d) Any compound including precursors or derivatives selected from raloxifen, tamoxifen, androgenic compounds, and their salts, where an intact phenol ring is present with a hydroxyl group present on carbons 1, 2, 3 and 4 of the terminal phenol ring.
(e) Any compound in the form of a prodrug that may be metabolized to form an active polycyclic-phenolic compound having bone protective activity.
   III. Methods for Using the Active Compounds

The active compounds which satisfy the criteria set out in detain herein can be used to treat a wide variety of medical conditions, including any condition in which it is helpful or necessary to build bone mass. Because of the discovery of the fundamental basis for bone loss (inappropriate osteoblastic apoptosis), one can for the first time envision the building of healthy bone as opposed to merely treating bone loss.

The active compounds can be used as bone anabolic agents in a host, including a human, to strengthen bone for strenuous physical activities such as sports or manual labor, and to strengthen bone in persons or other hosts who do not have osteoporosis but might be subject to osteoporosis in the future because the host is in a risk group for that disease. Other uses for a bone anabolic agent in humans include the treatment of hosts, including persons who are born with naturally thin, small, or unusually fragile bones, including weak teeth, persons who have a genetic predisposition to a bone catabolic disease, or an orthopedic bone disease such as joint degeneration, non-union fractures, orthopedic problems caused by diabetes, periimplantitis, poor responses to bone grafts, implants, or fracture.

These compounds can be used to increase the bone mass in horses and dogs used for labor as well as those used in sports such as racing. The compounds can also be used to increase the bone mass in chickens and turkeys used in meat production to increase the ease of processing.

Representative metabolic bone diseases are postmenopausal osteoporosis, senile osteoporosis in males and females, glucocorticoid-induced osteoporosis, immobilization-induced osteoporosis, weightlessness-induced osteoporosis (as in space flights), post-transplantation osteoporosis, migratory osteoporosis, idiopathic osteoporosis, juvenile osteoporosis, Paget's Disease, osteogenesis imperfecta, chronic hyperparathyroidism, hyperthyroidism, rheumatoid arthritis, Gorham-Stout disease, McCune-Albright syndrome and osteolytic metastases of various cancers or multiple myeloma. Characteristics of the orthopedic bone diseases are loss of bone mass, general bone fragility, joint degeneration, non-union fractures, orthopedic and dental problems caused by diabetes, periimplantitis, poor responses to bone grafts/implants/bone substitute materials, periodontal diseases, and skeletal aging and its consequences.

IV. Method for Screening for Compounds that Increase Bone Mass

The present invention provides a method of screening for compounds that possess bone anabolic effects, comprising the steps of: a) contacting a sample of osteoblast cells with a compound; and b) comparing the number of osteoblast cells undergoing apoptosis in the compound-treated cells with the number of osteoblast cells undergoing apoptosis in an untreated sample of osteoblast cells. A lower number of apoptotic cells following contact with the compound indicates that the compound possesses bone anabolic effects. Preferred compounds also inhibit apoptosis of osteocytes. Generally, the compound may be contacted with the sample either in vitro, e.g., in cell culture or in vivo, e.g., in an animal model. Typical methods of determining apoptosis are nuclear morphologic criteria, DNA end-labeling, DNA fragmentation analysis and immunohistochemical analysis.

In another embodiment, a method for selecting a compound that increases bone mass at least 10% in a host without a loss in bone strength or quality is provided that includes evaluating whether the compound (i) binds to the estrogen or androgen receptor (or the equivalent receptor in the host animal) with an association constant of at least 10⁸ M⁻¹ , and preferably, at least 10¹⁰ M⁻¹ : (ii) (a) induces estrogenic or androgenic gene transcriptional activity at a level that is no greater than 10% that of testosterone or 17β-estradiol, and preferably no greater than 5, 1 or even 0.1% that of 17β-estradiol or testosterone, as appropriate, when administered in vivo at a dosage of at least 0.1 ng/kg body weight or in vitro at concentrations of 10⁻¹¹ to 10⁻⁷ M in cells with the natural androgen or estrogen receptor or transfected with the androgen or estrogen receptor or (b) induces an increase in uterine or muscle weight or increase virilization in females, as appropriate, of no more than 10% that which is induced by 17β-estradiol or testosterone (or the equivalent compound in a host animal); (iii) induces the phosphorylation of extracellular signal regulated kinase (ERK) when administered in vivo at a dosage of at least 0.1 ng/kg body weight or in vitro in cells with the natural androgen or estrogen receptor or transfected with the androgen or estrogen receptor; and (iv) has an anti-apoptotic effect on osteoblasts at an in vivo dosage of at least 0.1 ng/kg body weight or in vitro in cells with the natural androgen or estrogen receptor or transfected with the androgen or estrogen receptor.

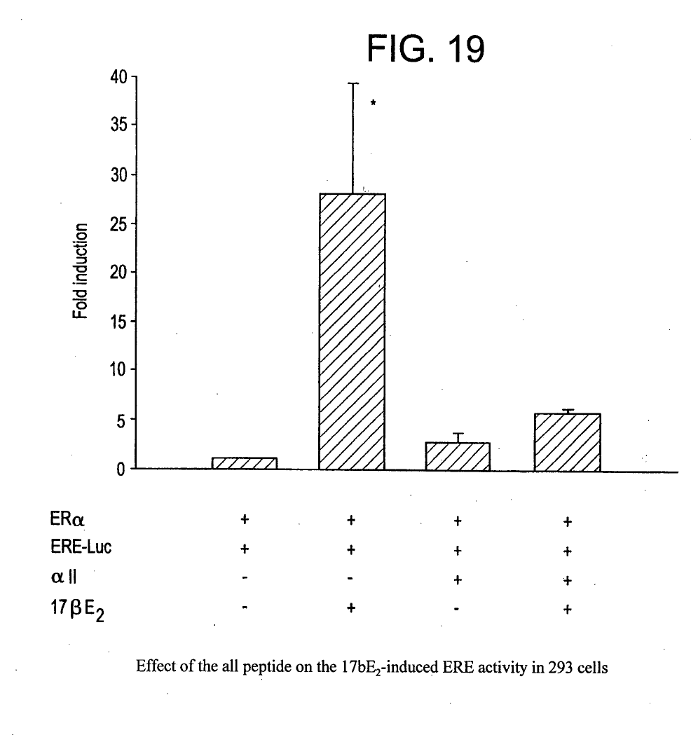

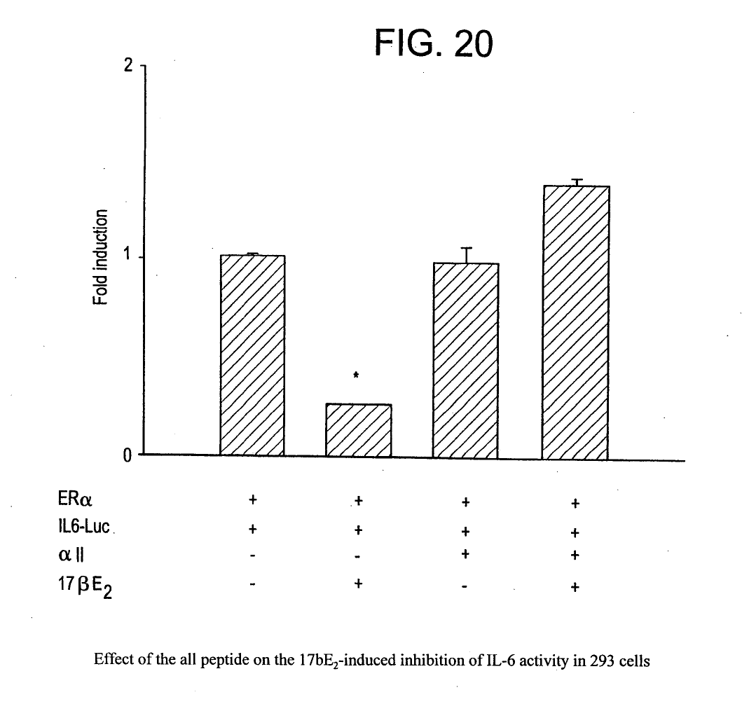

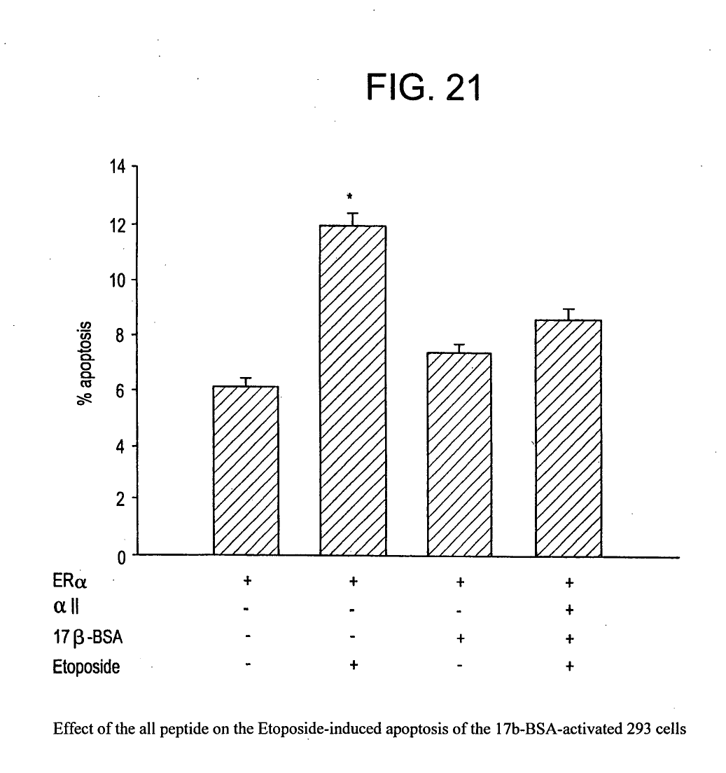

In another embodiment, a method for screening for compounds that bind to the estrogen or androgen receptor and activate the anti-apoptotic signalling pathway, without resultant transcriptional activation, is provided. This method is based on the fundamental discovery that the ligand-induced conformational changes of the estrogen receptor protein required for prevention of apoptosis, are distinct from the conformational changes required for transcriptional activity (FIGS. 19-21). This discovery allows for selecting compounds, from a large library of small molecules, which have anti-apoptotic, but not transcriptional, activity. Selection is accomplished using small peptides that can specifically block the transcriptional activity of ligand activated receptor, but do not interfere with the ability of the receptor to initiate the anti-apoptotic signalling cascade.

To accomplish this, cells are transfected with the estrogen or androgen receptor with or without a peptide that recognizes the conformation of the protein required for transcriptional activation, but not anti-apoptosis. Using this method, compounds that induce conformational changes resulting in both transcriptional and anti-apoptosis compatible conformations can be distinguished from compounds that only induce the latter conformational changes.

Nonlimiting examples of this method of screening include peptide binding assays for ERα or ERβ whereby the purified receptor protein is immobilized on streptavidin-coated plates using biotinylated vitellogenin ERE according to previously described methods of affinity selection (Sparks A B, Adey N B, Cwirla S, Kay B K Screening phage-displayed peptide libraries. In Phage Display of Peptides and Proteins, A Laboratory Manual, eds. Kay B K, Winter J and McCafferty J. (Academic, San Diego), pp.227-253, 1996). Following incubation with various ligands, the peptide is added and after 30 min bound peptide is detected using an anti-M13 antibody coupled to horseradish peroxidase. Compounds that bind to the receptor and induce conformational changes recognized by the peptide (i.e. the peptide binds to the receptor) will be discarded. The remaining compounds are then screened for anti-apoptotic potency.

V. Combination Therapy

In one aspect of the invention, one of the active compounds described herein can be administered to a host to increase bone mass in combination with a second pharmaceutical agent. The second pharmaceutical agent can be a bone anti-resorption agent, a second bone mass anabolizing agent, an antioxidant, a dietary supplement, or any other agent that increases the beneficial effect of the active compound on bone structure, strength, density, or mass.

Any member of the ten classes of drugs described in the Background of the Invention that are used in the treatment of osteoporosis can be administered in combination with the primary active agent, including: an anabolic steroid, a bisphosphonate, a calcitonin, an estrogen or progesterone, an anti-estrogens such as raloxifene or tamoxifene, parathyroid hormone (“PTH”), fluoride, Vitamin D or a derivative thereof, or a calcium preparations.

Nonlimiting examples of suitable agents for combination include, but are not limited to, alendronic acid, disodium clondronate, disodium etidronate, disodium medronate, disodium oxidronate, disodium pamidronate, neridronic acid, risedronic acid, teriparatide acetate, tiludronic acid, ipriflavone, potassium bicarbonate, progestogen, a thiazide, gallium nitrate, NSAIDS, plicamycin, aluminum hydroxide, calcium acetate, calcium carbonate, calcium, magnesium carbonate, and sucralfate.

Reducing agents, such as glutathione or other antioxidants may also be useful in combination with any of the compounds of the present invention. As used herein, the term antioxidant refers to a substance that prevents the oxidation of an oxidizable compound under physiological conditions. In one embodiment, a compound is considered an antioxidant for purposes of this disclosure if it reduces endogenous oxygen radicals in vitro. The antioxidant can be added to a cell extract under oxygenated conditions and the effect on an oxidizable compound evaluated. As nonlimiting examples, antioxidants scavenge oxygen, superoxide anions, hydrogen peroxide, superoxide radicals, lipooxide radicals, hydroxyl radicals, or bind to reactive metals to prevent oxidation damage to lipids, proteins, nucleic acids, etc. The term antioxidant includes, but is not limited to, the following classes of compounds:

A) Dithiocarbamates: Dithiocarbamates have been extensively described in patents and in scientific literature. Dithiocarbamates and related compounds have been reviewed extensively for example, by G. D. Thorn et al., entitled “The Dithiocarbamates and Related Compounds,” Elsevier, N.Y., 1962. Dithiocarboxylates are compounds of the structure, A-SC(S)-B, which are members of the general class of compounds known as thiol antioxidants, and are alternatively referred to as carbodithiols or carbodithiolates. It appears that the —SC(S)— moiety is essential for therapeutic activity, and that A and B can be any group that does not adversely affect the efficacy or toxicity of the compound. A and B can be selected by one of ordinary skill in the art to impart desired characteristics to the compound, including size, charge, toxicity, and degree of stability, (including stability in an acidic environment such as the stomach, or basic environment such as the intestinal tract). The selection of A and B will also have an important effect on the tissue-distribution and pharmacokinetics of the compound. The compounds are preferably eliminated by renal excretion.

B) N-Acetyl Cysteine and its Derivatives

Cysteine is an amino acid with one chiral carbon atom. It exists as an L-enantiomer, a D-enantiomer, or a racemic mixture of the L- and D-enantiomers. The L-enantiomer is the naturally occurring configuration.

N-acetylcysteine (acetamido-mercaptopropionic acid, NAC) is the N-acetylated derivative of cysteine. It also exists as an L-enantiomer, a D-enantiomer, an enantiomerically enriched composition of one of the enantiomers, or a racemic mixture of the L and D enantiomers. The term “enantiomerically enriched composition or compound” refers to a composition or compound that includes at least 95%, and preferably, at least 97% by weight of a single enantiomer of the compound. Any of these forms of NAC can be delivered as an antioxidant in the present invention. In one embodiment, a single isomer of a thioester or thioether of NAC or its salt, and most preferably, the naturally occurring L-enantiomer, is used in the treatment process.

N-acetylcysteine exhibits antioxidant activity (Smilkstein, Knapp, Kulig and Rumack, N. Engl. J. Med. 1988, Vol. 319, pp. 1557-62; Knight, K. R., MacPhadyen, K., Lepore, D. A., Kuwata, N., Eadie, P. A., O'Brien, B. Clinical Sci., 1991, Vol. 81, pp. 31-36; Ellis, E. F., Dodson, L. Y., Police, R. J., J. Neurosurg., 1991, Vol. 75, pp. 774-779). The sulfhydryl functional group is a well characterized, highly reactive free radical scavenger. N-acetylcysteine is known to promote the formation of glutathione (a tri-peptide, also known as g-glutamylcysteinylglycine), which is important in maintaining cellular constituents in the reduced state (Berggren, M., Dawson, J., Moldeus, P. FEBS Lett., 1984, Vol. 176, pp. 189-192). The formation of glutathione may enhance the activity of glutathione peroxidase, an enzyme which inactivates hydrogen peroxide, a known precursor to hydroxyl radicals (Lalitha, T., Kerem, D., Yanni, S., Pharmacology and Toxicology, 1990, Vol.66, pp. 56-61)

N-acetylcysteine exhibits low toxicity in vivo, and is significantly less toxic than deprenyl (for example, the LD₅₀ in rats has been measured at 1140 and 81 mg/kg intravenously, for N-acetylcysteine and deprenyl, respectively).

N-acetyl cysteine and derivatives thereof are described, for example, in WO/95/26719. Any of the derivatives described in this publication can be used in accordance with this invention.

C) Scavengers of Peroxides, including but not limited to catalase and pyruvate.

D) Thiols including dithiothreitol and 2-mercaptoethanol.

E) Antioxidants which are inhibitors of lipid peroxidation, including but not limited to Trolox™, BHA, BHT, aminosteroid antioxidants, tocopherol and its analogs, and lazaroids.

F) Dietary antioxidants, including antioxidant vitamins (vitamin C or E or synthetic or natural prodrugs or analogs thereof), either alone or in combination with each other, flavanoids, phenolic compounds, caratenoids, and alpha lipoic acid.

G) Inhibitors of lipoxygenases and cyclooxygenases, including but not limited to nonsteroidal antiinflammatory drugs, COX-2 inhibitors, aspirin-based compounds, and quercetin.

H) Antioxidants manufactured by the body, including but not limited to ubiquinols and thiol antioxidants, such as, and including glutathione, Se, and lipoic acid.

I) Synthetic Phenolic Antioxidants: inducers of Phase I and II drug-metabolizing enzymes.

VI. Pharmaceutical Compositions

An active compound or its pharmaceutically acceptable salt, selected according to the criteria described in detail herein, can be administered in an effective amount to treat any of the conditions described herein, optionally in a pharmaceutically acceptable carrier or diluent.

The active materials can be administered by any appropriate route for systemic, local or topical delivery, for example, orally, parenterally, intravenously, intradermally, subcutaneously, buccal, intranasal, inhalation, vaginal, rectal or topically, in liquid or solid form. Methods of administering the compound of the invention may be by specific dose or by controlled release vehicles.

A preferred mode of administration of the active compound is oral. Oral compositions will generally include an inert diluent or an edible carrier. The active compound can be enclosed in gelatin capsules or compressed into tablets. For the purpose of oral therapeutic administration, the compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable derivative or salts thereof can also be mixed with other active materials that do not impair the desired action, or with materials that supplement the desired action, such as classical estrogen like 17β-estradiol or ethinyl estradiol; bisphosphonates like alendronate, etidronate, pamidronate, risedronate, tiludronate, zoledronate, cimadronate, clodronate, ibandronate, olpadronate, neridronate, EB-1053; calcitonin of salmon, eel or human origin; and anti-oxidants like glutathione, ascorbic acid or sodium bisulfite. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS).

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted with monoclonal antibodies to surface antigens of specific cells) are also pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and/or cholesterol) in an inorganic solvent that is then evaporated, leaving behind a thin film of dried lipid on the surface of the container. An aqueous solution of the active compound or its monophosphate, diphosphate, and/or triphosphate derivative(s) is then introduced into the container. The container is then swirled by hand to free lipid material from the sides of the container and to disperse lipid aggregates, thereby forming the liposomal suspension.

The dose and dosage regimen will depend upon the nature of the metabolic bone disease, the characteristics of the particular active compound, e.g., its therapeutic index, the patient, the patient's history and other factors. The amount of an activator of non-genomic estrogen-like signaling compound administered will typically be in the range of about 1 pg/kg to about 10 mg/kg of patient weight. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press.

For parenteral administration, the active compound will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. An activator of non-genomic estrogen-like signaling compound will typically be formulated in such vehicles at concentrations of about 10 pg/ml to about 10 mg/ml.

The concentration of the compound in the drug composition will depend on absorption, inactivation, and excretion rates of the drug as well as other factors known to those of skill in the art. It is to be noted that dosage values will also vary with the severity of the condition to be alleviated. Additionally, the active ingredient may be administered at once, or may be divided into a number of smaller doses to be administered at varying intervals of time. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

VII. Illustrative Examples

The following examples are illustrations of the embodiments of the invention as described above, but are not intended to limit its scope.

As one example, 17β-estradiol, the synthetic steroid estratriene-3-ol, which is a potent neuroprotective compound, and 17α-estradiol, have potent anti-apoptotic effects on osteoblastic cells in vitro.

U.S. Pat. No. 5,843,934 to Simpkins discloses that an estrogen having insubstantial sex-related activity, and in particular, α-estrogens such as 17α-estradiol, can be administered to a patient to retard the adverse effects of osteoporosis in a male or female. The '934 patent does not address how to select a compound to increase bone mass opposed to treat osteoporosis. Increasing bone mass is a different indication from the treatment of bone loss, as dramatically illustrated by the fact that the U.S. Food and Drug Administration has approved a number of drugs for the treatment of osteoporosis, but has not approved any drugs to date as bone anabolic agents.

17β-Estradiol is used in these illustrative examples even though it is a potent activator of estrogen-like gene transcription, because it tightly binds to the estrogen receptor and inhibits osteoblastic apoptosis. The compound must be modified to fall within the selection criteria for the present invention by altering it in such a way that it cannot enter the cell to induce gene transcription. Such modifications can occur, for example, by covalently attaching, either directly or through a linking moiety, a second moiety that prevents or limits cell penetration. Any other estrogen or androgen that binds appropriately to the relevant receptor can be likewise modified for use to increase bone mass.

It is noteworthy that (a) the anti-apoptotic effect of 17β-estradiol on both osteoblasts and osteocytes are reproduced with a membrane impermeable 17β-estradiol-BSA conjugate; (b) the anti-apoptotic effects of these compounds are diminished by ICI 182780, a pure estrogen receptor antagonist; and (c) that the anti-apoptotic effects of all these compounds cannot be shown in HeLa cells unless these cells are stably transfected with either the estrogen receptor α or the estrogen receptor β.

The following examples are given for the purpose of illustrating various embodiments of the invention and are not meant to limit the present invention in any fashion.

### EXAMPLE 1

The increased rate of bone remodeling that follows loss of estrogen should cause a transient acceleration of mineral loss because bone resorption is faster than bone formation and the bone made by new BMUs are less dense than older ones. However, increased remodeling alone cannot explain the progressive bone loss that lasts long after the rate of bone remodeling has slowed. Indeed, in addition to changes in the number of osteoblast and osteoclast cells during/following estrogen deficiency, a qualitative abnormality also occurs; osteoclasts erode deeper than normal cavities. This frequently leads to penetration through a trabecular structure causing removal of some cancellous elements entirely; the remainder are more widely separated and less well connected. The deeper erosion is explained by loss of estrogen's effect to promote apoptosis of osteoclasts (Hughes et al, Nature Med. 1996; 2:1132-1136; Kameda et al, J Exp Med. 1997; 186:489-495; Raisz, Nature Med. 1996; 2:1077-1078). 17β-estradiol increased the apoptosis of osteoclasts from approximately 0.5% to as much as 2.7%. This change could prolong the lifespan of osteoclasts and increase their numbers two- to three-fold, thus accounting for the perforation of trabeculae and grinding away of endocortical margins.

To determine whether the role of estrogen deficiency affects osteoblast and osteocyte apoptosis, the prevalence of these cells in murine vertebrae removed 28 days after ovariectomy was determined. In these experiments, four month old Swiss Webster mice were ovariectomized and 28 days later, the animals were sacrificed and the vertebrae were isolated, fixed and embedded undecalcified in methacrylate. As shown in FIG. 2, the prevalence of osteoblast and osteocyte apoptosis, determined by TUNEL with CuSO₄ enhancement, increased ten- and four-fold, respectively. These results indicate that the accelerated loss of bone that occurs after estrogen deficiency is due not only to an increase in osteoclast number and lifespan, but also to a premature reduction in the lifespan (work hours) of the osteoblasts. The increase in osteocyte apoptosis could further weaken the skeleton by impairment of the osteocyte-canalicular mechanosensory network.

### EXAMPLE 2

Consistent with the in vivo data described under Example 1, 17β-estradiol prevented apoptosis of osteoblastic cells isolated from murine calvaria, in a dose dependent manner. Strikingly, inhibition of osteoblast apoptosis could also be shown by 17β-estradiol conjugated with bovine serum albumin, a membrane impermeable compound. The same effect could also be shown with 17α-estradiol, a compound heretofore thought to be inactive. Moreover, inhibition of etoposide-induced osteoblastic cell apoptosis was demonstrated by estratriene-3-ol, an estrogenic compound thought to lack feminizing properties (FIG. 3). In this experiment, osteoblastic cells were derived from murine calvaria and were pretreated with the sterols for 1 hour before the addition of the pro-apoptotic agent, etoposide.

### EXAMPLE 3

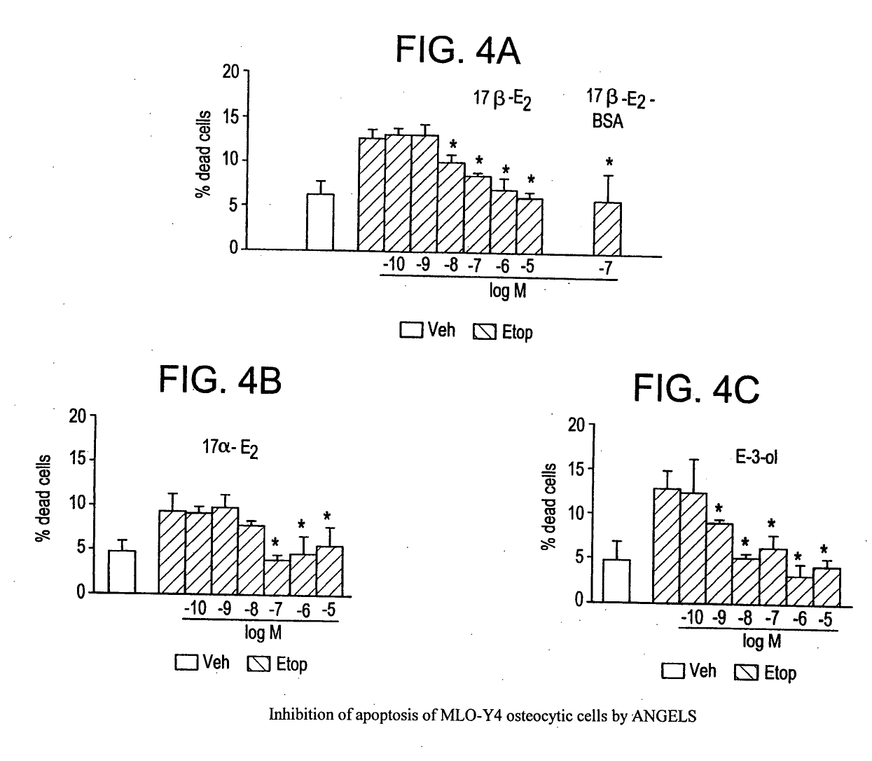

In agreement with the in vivo results indicating that estrogen loss increases both osteoblast and osteocyte apoptosis, 17β-estradiol, 17β-estradiol conjugated with BSA, 17α-estradiol, and estratriene-3-ol dose-dependently inhibited also the apoptosis of an established osteocytic cell line (FIG. 4). In this experiment, MLO-Y4 cells were pretreated with the indicated concentrations of the various compounds for 1 h before the addition of the pro-apoptotic agent, etoposide. Apoptosis was determined after 6 h by trypan blue uptake as described in FIG. 3.

### EXAMPLE 4

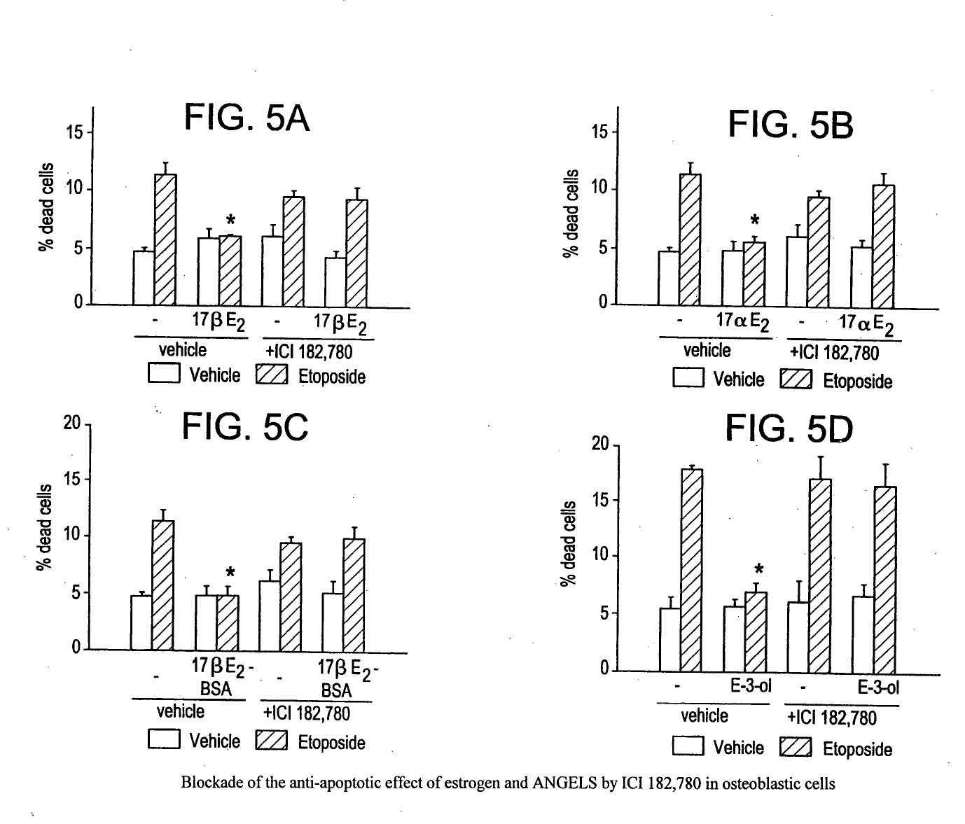

As shown in FIG. 5, the anti-apoptotic effect of 10⁻⁸ M 17β-estradiol, 17β-estradiol-BSA, 17α-estradiol, or estratriene-3-ol (E-3-ol) on osteoblastic cells was abrogated when the cells were pretreated for 1 h with the pure receptor antagonist ICI182,780 (10⁻⁷ M) before the addition of the estrogenic compounds.

### EXAMPLE 5

As in the case of the antiapoptotic effect of 17β-estradiol, 17β-estradiol-BSA, 17α-estradiol, or estratriene-3-ol (E-3-ol) on osteoblastic cells, their antiapoptotic effect on osteocytes was abrogated when the cells were pretreated for 1 h with the pure receptor antagonist ICI182,780 (10⁻⁷ M). Collectively, the results of examples 4 and 5 strongly suggest that the anti-apoptotic effects of these compounds on osteoblasts and osteocytes are mediated via the estrogen receptor (ER).

### EXAMPLE 6

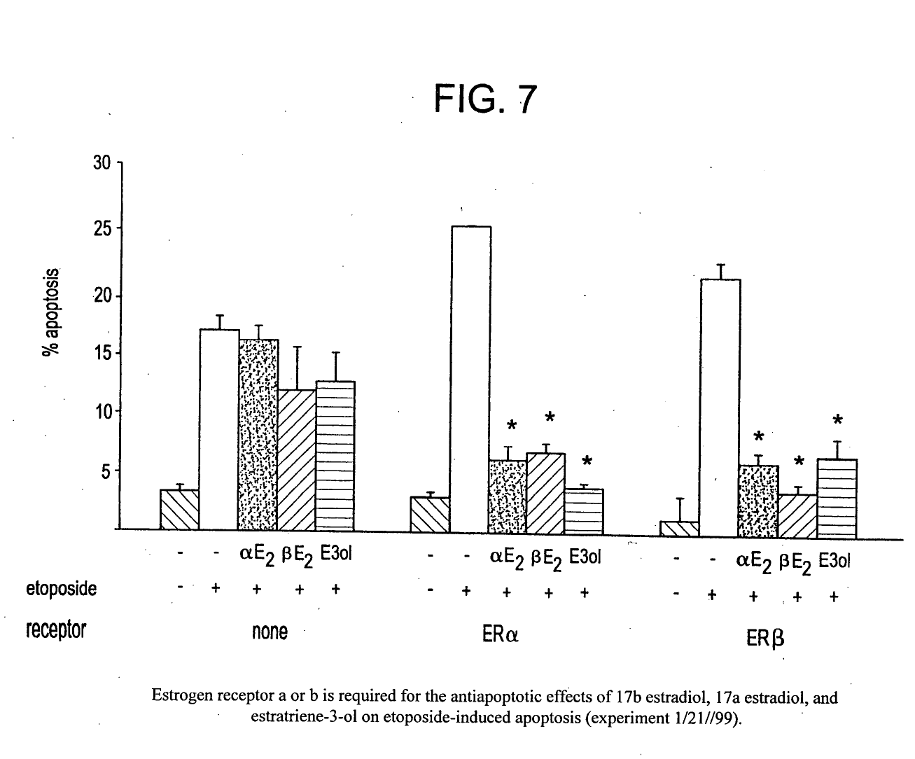

Definitive demonstration of the requirement of the estrogen receptor for the anti-apoptotic effects of 17β-estradiol and the related compounds tested herein was provided by the results of the experiment shown in FIG. 7. In this experiment, instead of calvaria cells, human HeLa cells which contain undetectable, if any, estrogen receptor were used. HeLa cells were stably transfected with either a CMV promoter-driven cDNA for the murine estrogen receptor-alpha (mERα) or a CMV promoter-driven cDNA for the murine estrogen receptor-beta (mERβ). Subconfluent cultures of stable transfectants were treated for 1 h with 17β-estradiol, or 17α-estradiol, estratriene-3-ol (10⁻⁸ M), followed by a 6 hour incubation with etoposide (5×10⁻⁵ M). Cells were trypsinized, pelleted and trypan blue positive cells were enumerated. As shown in FIG. 7, none of the three compounds had any effect on the apoptosis of the wild type HeLa cells, but they potently inhibited etoposide-induced apoptosis in HeLa cells transfected with the estrogen receptor α or estrogen receptor β.

### EXAMPLE 7

The mechanism of the anti-apoptotic effect of the estrogenic compounds described herein was established by demonstrating that 17α-estradiol, 17β-estradiol, 17β-estradiol-BSA or estratriene-3-ol, at 10⁻⁸ M concentrations, activated extracellular signal regulated kinases (ERKs). In this experiment, MLO-Y4 osteocytic cells were incubated for 25 minutes in serum-free medium. Subsequently, 17α-estradiol, 17β-estradiol, 17β-estradiol-BSA or estratriene-3-ol (10⁻⁸ M) were added and cells incubated for an additional 5, 15, or 30 minutes. Cell lysates were prepared and proteins were separated by electrophoresis in polyacrylamide gels and transferred to PVDF membranes. Western blotting was performed using a specific antibody recognizing phosphorylated extracellular signal regulated kinases 1 and 2, followed by reblotting with an antibody recognizing total extracellular signal regulated kinases. Blots were developed by enhanced chemiluminescence. As shown in FIG. 8, all these compounds specifically increased the phosphorylated fraction of ERK1/2 without affecting the total amount of ERK1/2. This effect is too rapid to be accounted for by the classical mechanism of estrogen action. Instead, it is consistent with a non-genomic action mediated via membrane-associated estrogen receptors, as suggested by the experiments presented in Examples 4, and 6.

### EXAMPLE 8

The ability of 17α-estradiol, 17β-estradiol, 17β-estradiol-BSA or estratriene-3-ol to activate ERKs was abrogated in the presence of the specific inhibitor of ERK kinase, PD98059. In this experiment, MLO-Y4 osteocytic cells were incubated for 25 minutes in serum-free medium in the presence or absence of 50 μM PD98059. Subsequently, 17α-estradiol, 17β-estradiol, 17β-estradiol-BSA or estratriene-3-ol (10⁻⁸ M) were added and cells incubated for another 5 minutes. Cell lysates were prepared and proteins were separated by electrophoresis in polyacrylamide gels and transferred to PVDF membranes. Western blotting was performed using a specific antibody recognizing phosphorylated extracellular signal regulated kinases 1 and 2, followed by reblotting with an antibody recognizing total extracellular signal regulated kinases. Blots were developed by enhanced chemiluminescence.

### EXAMPLE 9

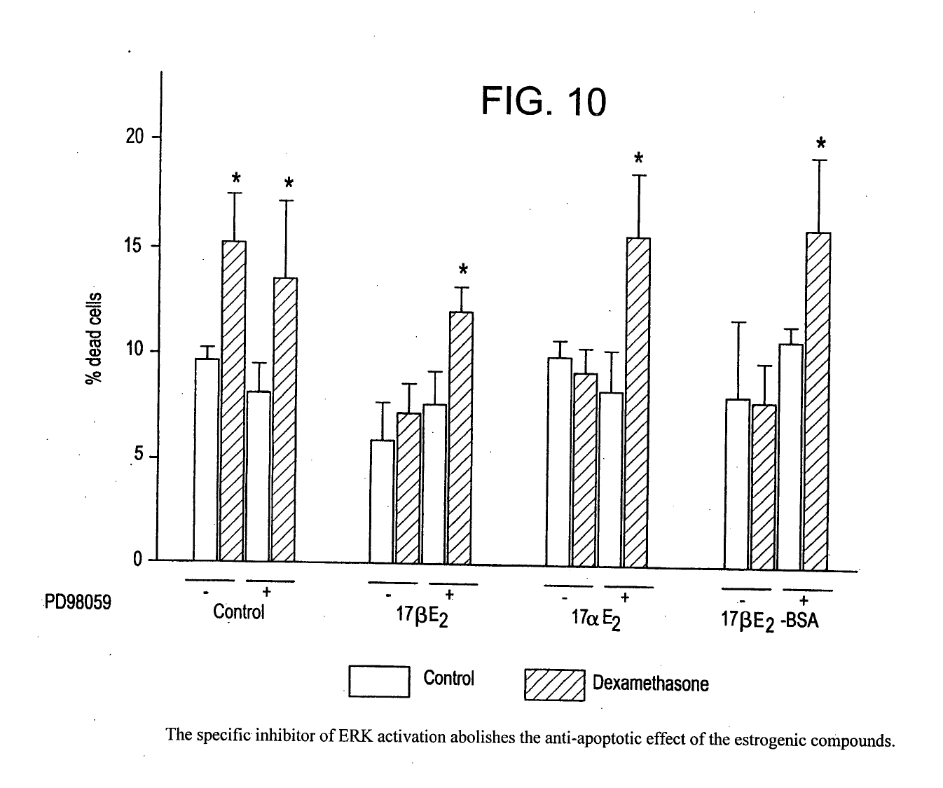

That indeed the anti-apoptotic effect of all the compounds tested herein was mediated via activation of ERKs was established by the results of the experiments shown in FIG. 10. In this experiment, MLO-Y4 osteocytic cells were pretreated for 1 hour with the specific inhibitor of ERKs activation, PD98059, before the addition of 10⁻⁸ M 17α-estradiol, 17β-estradiol, or 17β-estradiol-BSA. Apoptosis was induced by incubation with the pro-apoptotic agent dexamethasone for 6 hours and quantified as described in FIG. 3. PD98059 prevented the anti-apoptotic effect of all three compounds tested in this experiment.

In conclusion, the results of the examples provided above demonstrate that loss of estrogen in vivo leads to several-fold increase in the prevalence of apoptosis of osteoblasts and osteocytes. Consistent with the in vivo findings, 17α-estradiol, as well as 17β-estradiol, 17β-estradiol-BSA and estratriene-3-ol inhibit the apoptosis of osteoblastic cells derived from murine calvaria or osteocytes, represented herein by the cell line MLO-Y4. The anti-apoptotic effect of all these compounds requires the presence of either estrogen receptor α or estrogen receptor β and is mediated via the ability of these compounds to activate specific MAP kinases, namely the extracellular signal regulated kinases (ERKs).

### EXAMPLE 10

Similar to the results with estrogenic compounds, androgenic compounds also inhibited apoptosis of osteoblastic cells derived from murine calvaria induced by etoposide (Table 3). In these experiments, cells were pretreated with the indicated concentrations of the various compounds for 1 hour, in the absence or presence of the androgen receptor antagonist flutamide, before the addition of the proapoptotic agent etoposide. Apoptosis was determined after 6 hours by trypan blue uptake as described in FIG. 3. Notably, as in the case of estrogenic compounds, all these effects were apparently mediated by the androgen receptor, as evidenced by the inhibition of the anti-apoptotic effects of the androgenic compounds by a specific androgen receptor antagonist. Moreover, and as in the case of estrogens, the androgen receptor-mediated protection of etoposide-induced apoptosis was seen with a membrane impermeable androgen (testosterone-17β-hemisuccinate conjugated with BSA), strongly suggesting the existence of a membrane-associated androgen receptor, analogous to the membrane-associated estrogen receptor.

TABLE 3
Inhibition of etoposide-induced osteoblast apoptosis
by androgens and progestins

| | Lowest | |
|---|---|---|
| | Effective | Suppression by 10⁻⁸ M |
| Compound | Concentration | Flutamide |
| Testosterone | 10⁻⁹ M | yes |
| Testosterone 17β- | 10⁻⁸ M | yes |
| Hemisuccinate: BSA | | |
| 5-α- | 10⁻⁹ M | yes |
| dihydrotestosterone | | |
| 5-β- | 10⁻¹⁰ M | yes |
| dihydrotestosterone | | |
| Dehydroisoandrosterone- | 10⁻⁸ M | no* |
| 3-sulfate | | |
| (DHES) | | |
| 4-androstene-3,17- | 10⁻⁸ M | yes |
| dione | | |
| 5-androstene-3β- | 10⁻⁸ M | yes |
| 17_-diol | | |
| RU1881 | 10⁻⁸ M | yes |

*Flutamide did block the anti-apoptotic effect of DHES at higher (10⁻⁷ M) concentration.

### EXAMPLE 11

That the anti-apoptotic effects of estrogenic compounds is dissociated from their transcriptional activity was established by demonstrating that even though estratriene-3-ol was as potent as 17β estradiol in inhibiting apoptosis, unlike 17β estradiol, it did not transactivate an estrogen response element through the estrogen receptor α. In this experiment, hERα was overexpressed in 293 cells (which lack constitutive ERα) along with a reporter construct containing 3 copies of an estrogen response element driving the luciferase gene. Light units were counted and normalized to coexpressed β-galactosidase activity to control for differences in transfection efficiency.

### EXAMPLE 12

Herein, a general experimental protocol for studies aiming to evaluate compounds with anti-apoptotic efficacy, but decreased transcriptional activity (e.g., estratriene-3-ol) on osteoblasts and osteocytes in animal models is provided. According to this design, estrogen-replete or estrogen-deficient mice, rats, dogs, primates, etc., or animals representing models of involutional osteoporosis and/or defective osteoblastogenesis (e.g., the senescence accelerated mouse, SAMP6: (Jilka et al., J Clin Invest 97:1732-1740, 1996)), or animal models of glucocorticoid excess (e.g., Weinstein et al. J Clin Invest, 102:274-282, 1998) are administered estratriene-3-ol or other test compound to determine whether they can suppress osteoblast and osteocyte apoptosis and whether changes in apoptosis would be associated with changes in BMD, bone formation rate, or cancellous bone volume.

In a representative experiment of this sort, six 4-5 month old female mice per group are screened twice for BMD in a four week period immediately prior to the initiation of the experiment to establish that peak adult bone mass has been attained. A subset of mice are then ovariectomized. Intact and ovariectomized mice are treated with vehicle, or 20, 200 or 2000 ng/g body weight estratriene-3-ol or another test compound. Ovariectomized mice are also treated with 20 ng/g body weight 17β-estradiol for comparison purposes.

Stock solutions of the test agents (10,000 μg/ml) are maintained in approximately 2.0 ml of 95% ethanol. These stocks are diluted in 95% ethanol to make 1000 μg/ml and 100 μg/ml concentrations. The concentration of the stocks is checked spectrophotometrically. For each animal injection, the test agent is diluted in sesame oil and sonicated. Test agents are administered for 28 days by subcutaneous injections on alternative days. The mice are weighed weekly and serum samples are collected at appropriate times for analysis of bone biochemical markers, such as osteocalcin or collagen cross-links. Tetracycline labeling is performed by administration of the antibiotic (30 mg/kg) at 2 and 8 days prior to the end of each experiment. Table 1 shows a representative example of 25 g mice divided into 5 groups with each animal receiving 100 μl of the test agent per injection.

TABLE 1

| | Injection |
|---|---|
| Treatment | (steroid + sesame oil) |
| vehicle | 100 μl 95% ethanol + 1900 μl |
| 20 ng/g estratriene-3-ol | 100 μl 100 μg/ml stock + 1900 μl |
| 200 ng/g estratriene-3-ol | 100 μl 1000 μg/ml stock + 1900 μl |
| 2000 ng/g estratriene-3-ol | 100 μl 10,000 μg/ml stock +1900 μl |
| 20 ng/g 17β-estradiol | 50 μl 100 μg/ml stock + 950 μl |

During the 28 day experiment, BMD is determined in live animals at day 0, 14 and 28. Following animal sacrifice at the end of the experiment, the vertebral bones L1-L4 are collected for fixation and embedded undecalcified in methylmethacrylate plastic for the determination of the prevalence of osteoblast and osteocyte apoptosis and other static and dynamic histomorphometric measurements. L5 vertebrae are isolated for determining anti-fracture efficacy of the compounds by assaying compression, 3 point bending and other appropriate biomechanical tests. Results confirming the expected efficacy of these compounds show decreased prevalence of osteoblast and/or osteocyte apoptosis, and/or positive BMD changes, and/or increased cancellous bone area, and/or increased rate of bone formation, and/or increased biomechanical strength.

As an example, the results of an experiment whereby 2000 ng/g body weight of estratriene-3-ol was administered for 28 days to estrogen-replete (intact) or estrogen-deficient (ovariectomized) mice are shown in Table 2.

TABLE 2
Increased BMD by estratriene-3-ol administration

| | | | | | |
|---|---|---|---|---|---|
| | 1 | | | | |
| global | hindquart | spine 1 | global | 2 hindquart | spine 2 |

intact-vehicle:

| | | | | | | |
|---|---|---|---|---|---|---|
| 0.0552 | 0.0585 | 0.0599 | 0.0533 | 0.0581 | 0.0595 | |
| 0.0535 | 0.0586 | 0.0575 | 0.0546 | 0.0571 | 0.0599 | |
| 0.0516 | 0.0557 | 0.0559 | 0.0503 | 0.0560 | 0.0544 | |
| 0.0516 | 0.0513 | 0.0569 | 0.0492 | 0.0499 | 0.0527 | |
| 0.0552 | 0.0553 | 0.0589 | 0.0492 | 0.0521 | 0.0531 | |
| 0.0475 | 0.0494 | 0.0525 | 0.0480 | 0.0450 | 0.0539 | |
| 0.0535 | 0.0524 | 0.0574 | 0.0524 | 0.0592 | 0.0553 | |
| 0.0526 | 0.0544 | 0.0570 | 0.0510 | 0.0539 | 0.0555 | (mean) |
| 0.0027 | 0.0036 | 0.0024 | 0.0025 | 0.0052 | 0.0030 | (std) |
| 0.0552 | 0.0586 | 0.0599 | 0.0546 | 0.0592 | 0.0599 | (max) |
| 0.0475 | 0.0494 | 0.0525 | 0.0480 | 0.0450 | 0.0527 | (min) |

intact-2000 ng/g 3-ol:

| | | | | | | |
|---|---|---|---|---|---|---|
| 0.0486 | 0.0509 | 0.0511 | 0.0550 | 0.0585 | 0.0602 | |
| 0.0511 | 0.0565 | 0.0560 | 0.0543 | 0.0603 | 0.0604 | |
| 0.0543 | 0.0605 | 0.0593 | 0.0541 | 0.0619 | 0.0601 | |
| 0.0537 | 0.0577 | 0.0584 | 0.0568 | 0.0629 | 0.0613 | |
| 0.0533 | 0.0546 | 0.0571 | 0.0568 | 0.0640 | 0.0620 | |
| 0.0521 | 0.0544 | 0.0555 | 0.0537 | 0.0554 | 0.0588 | |
| 0.0560 | 0.0587 | 0.0623 | 0.0574 | 0.0605 | 0.0632 | |
| 0.0527 | 0.0562 | 0.0571 | 0.0554 | 0.0605 | 0.0609 | (mean) |
| 0.0024 | 0.0032 | 0.0035 | 0.0015 | 0.0029 | 0.0014 | (std) |
| 0.0560 | 0.0605 | 0.0623 | 0.0574 | 0.0640 | 0.0632 | (max) |
| 0.0486 | 0.0509 | 0.0511 | 0.0537 | 0.0554 | 0.0588 | (min) |

vehicle vs 2000 ng/g:

| | | | |
|---|---|---|---|
| t-test | 0.0036 | 0.0077 | 0.0037 |

Ovx-vehicle:

| | | | | | | |
|---|---|---|---|---|---|---|
| 0.0525 | 0.0539 | 0.0550 | 0.0493 | 0.0507 | 0.0548 | |
| 0.0479 | 0.0484 | 0.0538 | 0.0497 | 0.0569 | 0.0545 | |
| 0.0510 | 0.0543 | 0.0565 | 0.0483 | 0.0509 | 0.0545 | |
| 0.0538 | 0.0548 | 0.0583 | 0.0483 | 0.0515 | 0.0536 | |
| 0.0567 | 0.0632 | 0.0620 | 0.0538 | 0.0584 | 0.0588 | |
| 0.0533 | 0.0533 | 0.0572 | 0.0504 | 0.0507 | 0.0559 | |
| 0.0543 | 0.0592 | 0.0583 | 0.0491 | 0.0526 | 0.0545 | |
| 0.0490 | 0.0518 | 0.0551 | 0.0475 | 0.0487 | 0.0534 | |
| 0.0523 | 0.0550 | 0.0570 | 0.0496 | 0.0528 | 0.0550 | (mean) |
| 0.0029 | 0.0049 | 0.0026 | 0.0019 | 0.0035 | 0.0017 | (std) |
| 0.0567 | 0.0632 | 0.0620 | 0.0538 | 0.0584 | 0.0588 | (max) |

Ovx-2000 ng/g 3-ol:

| | | | | | | |
|---|---|---|---|---|---|---|
| 0.0505 | 0.0527 | 0.0547 | 0.0565 | 0.0602 | 0.0608 | |
| 0.0542 | 0.0588 | 0.0581 | 0.0557 | 0.0632 | 0.0585 | |
| 0.0496 | 0.0504 | 0.0542 | 0.0548 | 0.0599 | 0.0584 | |
| 0.0540 | 0.0596 | 0.0586 | 0.0545 | 0.0624 | 0.0598 | |
| 0.0526 | 0.0547 | 0.0580 | 0.0564 | 0.0598 | 0.0610 | |
| 0.0569 | 0.0604 | 0.0628 | 0.0568 | 0.0647 | 0.0625 | |
| 0.0565 | 0.0591 | 0.0603 | 0.0550 | 0.0630 | 0.0578 | |
| 0.0528 | 0.0582 | 0.0568 | 0.0539 | 0.0599 | 0.0605 | |
| 0.0534 | 0.0573 | 0.0579 | 0.0555 | 0.0618 | 0.0599 | (mean) |
| 0.0026 | 0.0036 | 0.0028 | 0.0011 | 0.0020 | 0.0016 | (std) |
| 0.0569 | 0.0604 | 0.0628 | 0.0568 | 0.0647 | 0.0625 | (max) |
| 0.0496 | 0.0504 | 0.0542 | 0.0539 | 0.0598 | 0.0578 | (min) |

ovx vs 2000 ng/g:

| | | | |
|---|---|---|---|
| t-test | 0.0000 | 0.0001 | 0.0001 |

Each row represents values for individual animals. The first three sets of numbers represent the initial BMD measurements (by dual-energy x-ray absorptiometry with Hologic QDR2000 plus, using customized software) at day 0 and the last three BMD measurements at the end of the experiment. Global=BMD of the entire skeleton minus the head and tail; hindquarters=the mean BMD of both hindlimbs; spine=the BMD of cervical, thoracic and lumbar spine.

### EXAMPLE 13

Herein, a general experimental protocol evaluating the anti-fracture efficacy of compounds like estratriene-3-ol is provided. According to this design, estrogen-replete or estrogen-deficient mice, rats, dogs, primates, etc., or animals representing models of involutional osteoporosis and/or defective osteoblastogenesis (e.g., the senescence accelerated mouse, SAMP6: (Jilka et al., J Clin Invest 97:1732-1740, 1996)), or animal models of glucocorticoid excess (e.g., Weinstein et al. J Clin Invest, 102:274-282, 1998) are administered estratriene-3-ol to determine whether they can increase bone strength.

In a representative experiment of this sort, seven 4-5 month old female mice per group are screened twice for BMD in a four week period immediately prior to the initiation of the experiment to establish that peak adult bone mass has been attained. A subset of mice are then ovariectomized. Intact and ovariectomized mice are treated with vehicle, or 20, 200 or 2000 ng/g body weight estratriene-3-ol or another ANGEL compound. Ovariectomized mice are also treated with 20 ng/g body weight 17β-estradiol for comparison purposes. Ultimate load bearing properties of the fifth lumbar murine vertebrae (L5) is determined. This is done using a servohydraulic axial-torsional material testing machine (Model MTS 810 Bionx; MTS Systems Corp., Eden Prairie, Minn.) and a Lebow load cell (Eaton Products, Troy, Mich.). Data are recorded and analyzed using the LabVIEW software package and an acquisition/signal conditioning board (Model NB-MIO-16, National Instruments Corporation, Austin, Tex.). The L5 specimens that is used for ultimate load bearing is cleaned of surrounding soft tissue and the length and diameter recorded with a digital caliper at a resolution of 0.01 mm (Mitutoyo Model #500-196, Ace Tools, Ft. Smith, Ark.). The vertebrae are wrapped in saline-soaked gauge throughout preparation and testing and stored overnight at 4° C. before testing. Vertebrae are individually compressed between parallel loading platens along the cephalocaudad axis until failure and the ultimate load (in Newtons) and displacement (in mm) are recorded.

As an example, the results of an experiment whereby 2000 ng/g body weight of estratriene-3-ol was administered for 28 days to estrogen-replete (intact) or estrogen-deficient (ovariectomized) mice (from the same animals shown in Example 12) is shown in Table 4.

TABLE 4
Changes in Vertebral Compression Strength (VCS*),
Induced by In vivo Administration of E-3-ol:
Demonstration of Greater Increase in CVS than BMD
(n = 7 per group)

| | Vertebral | |
|---|---|---|
| | Compression | |
| | (Newtons) | Global BMD (g/cm2) |
| Intact- | 66.78 ± 17.47 | 0.0508 ± 0.0026 |
| vehicle | | |
| Ovx- | 50.3 ± 7.58 | 0.0486 ± 0.0011 |
| vehicle | | |
| Intact-E- | 96.26 ± 15.92 | 0.0554 ± 0.0015 |
| 3-ol | (p < 0.006) | (p < 0.002) |
| Ovx-E-3- | 85.57 ± 10.17 | 0.0555 ± 0.0011 |
| ol | (p < 0.00001) | (p < 0.00001) |

*Each value represents the mean from seven animals. The BMD values shown for comparison here are from the experiment described in Example 12.

### EXAMPLE 14

To determine whether the anti-apoptotic effects of estrogenic compounds are mechanistically dissociable from their transcriptional effects, specific conformational changes of the receptor protein leading to prevention of apoptosis versus transcriptional activity were sought. The rationale behind these studies was based on recent evidence that the transcriptional activity of the ER is greatly dependent on ligand-induced conformational changes of the receptor protein. Indeed, using phage display libraries, McDonnell and co-workers have recently screened for and isolated four classes of small (11 amino acids) peptides that recognize distinct conformational changes of the estrogen receptor, and can either selectively block transcription from specific ligands (e.g., estradiol but not tamoxifen and vice versa) or selectively block ERα but not ERβ-mediated transcription, and vice versa, when tested on a consensus ERE (Norris et al. Science 285:744-746, 1999). The first class contains the LXXLL motif and can interact with both estradiol-activated β and ERα. The second class displays specific interaction with estradiol- and tamoxifen-activated ERα, whereas the third class can interact specifically with tamoxifen-activated ERβ. Yet a fourth class with a SREWFXXXL conserved motif was found to complex to tamoxifen-activated ERα and ERβ. Indeed, when fusion proteins made with these peptides and the Gal4-DNA binding domain and were co-expressed with ER in HeLa cells they functioned as ligand-receptor complex-specific antagonists, demonstrating that ligand activation triggers transcriptional activity by conferring specific conformational changes on the receptor protein (Paige L A, Christensen D J, Gron H, Norris J D, Gottlin E B, Padilla K M, Change C-Y, Ballas L M, Hamilton P T, McDonnell DP, Fowlkes D M. Estrogen receptor (ER) modulators each induce distinct conformational changes in β and ERβ. Proc. Natl. Acad. Sci 96:3999-4004, 1999).

Based on the findings that estrogenic compounds like the conjugated 17-β estradiol with BSA have, at least as potent anti-apoptotic effects as estrogen while have significantly decreased transcriptional activity, the hypothesis that the non-genomic anti-apoptotic effects of estrogen can be initiated by distinct ligand-dependent conformational changes of the ER, as compared to the conformational changes required for the transcriptional effects of the ER was tested. It was found that indeed there is dissociation of conformational changes. Based on this, one can explain the mechanistic basis of the apparent dissociation of the two sets of actions. This knowledge forms the basis for the design of the screening strategies described herein for ligands which display non-transcriptional effects, but lack the ability to initiate transcriptional activation.

One skilled in the art will readily appreciate that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1-45. (canceled)

46. A method of screening for a compound that increases bone mass in a host at least 10% without a loss in bone strength or quality, comprising:
(a) determining that the compound includes the following characteristics:
(i) binds to an estrogen α or β like receptor with an association constant of at least 10⁸ M⁻¹;
(ii)(a) induces estrogenic gene transcriptional activity at a level that is no greater than 10% that of 17β-estradiol when administered in vivo at concentrations of 10¹¹ to 10⁻⁷ M and at a dosage of at least 0.1 ng/kg body weight or in vitro in osteoblastic or osteocytic cells with estrogen receptors or (b) induces an increase in uterine weight of no more than 10% that of 17β-estradiol or analogs thereof;
(iii) induces the phosphorylation of extracellular signal regulated kinase (ERK) when administered in vivo at a dosage of at least 0.1 ng/kg body weight or in vitro at concentrations of 10⁻¹¹ to 10⁻⁷ M in osteoblastic cells with estrogen receptors; and
(iv) has an anti-apoptotic effect on osteoblasts at an in vivo dosage of at least 0.1 ng/kg body weight or in vitro in osteoblastic or osteocytic cells with natural estrogen receptors or cells transfected with estrogen receptors; and
(b) selecting said compound including said characteristics.

47. The method of claim 46, wherein determining that the compound binds to an estrogen α or β like receptor with an association constant of at least 10⁸ M⁻¹ comprises: contacting the compound with an estrogen receptor from the host and measuring the association constant.

48. The method of claim 47, wherein the association constant of is measured by a competitive radiometric binding assay, comprising:
a) contacting the compound with a sample comprising estrogen receptors and [³H] estradiol,
b) incubating at room temperature or overnight at 4° C., allowing the binding of the compound to the receptor;
c) isolating bound receptor; and
d) comparing the amount of bound receptor displaced by the compound to a control sample of estrogen receptors with [³H] estradiol to determine the association constant.

49. The method of claim 46, wherein the compound binds with an association constant of at least 10¹⁰M⁻¹.

50. The method of claim 46, wherein determining that the compound induces estrogenic gene transcriptional activity at a level that is no greater than 10% that of 17β-estradiol when administered in vivo at concentrations of 10⁻¹¹ to 10⁻⁷ M and at a dosage of at least 0.1 ng/kg body weight comprises: administering the compound to a host and monitoring the level of induction or suppression of a surrogate marker of estrogenic transcriptional activity.

51. The method of claim 50, wherein the surrogate marker of is selected from the group consisting of: complement C3 gene, lactoferin, IL-6 and a minimal gene containing one or more copies of the ERE driving a reporter gene.

52. The method of claim 46, wherein determining that the compound induces estrogenic gene transcriptional activity at a level that is no greater than 10% that of 17β-estradiol when administered in vitro at concentrations of 10⁻¹¹ to 10⁻⁷ M and at a dosage of at least 0.1 ng/kg body weight includes: contacting the compound with cells having estrogen receptors and monitoring the level of induction or suppression of a surrogate marker.

53. The method of claim 52, wherein the surrogate marker of is selected from the group consisting of: complement C3 gene, lactoferin, IL-6 and a minimal gene containing one or more copies of the ERE driving a reporter gene.

54. The method of claim 53, wherein the cells of are selected from the group consisting of: human uterine HeLa cells, human embryonic kidney cells 293, murine osteocytic MLO-Y4 cells and murine osteoblastic calvaria derived cells.

55. The method of claim 46, wherein the determining of uterine weight increase includes ultrasound.

56. The method of claim 46, wherein the determining of uterine weight increase includes removing the uterus, weighing the uterus and comparing uterine weight to total body weight.

57. The method of claim 46, wherein determining the induction of the phosphorylation of extracellular signal regulated kinase (ERK) when administered in vivo at a dosage of at least 0.1 ng/kg body weight includes administering the compound to the host and measuring the increase of phosphorylation of ERK.

58. The method of claim 46, wherein determining the induction of the phosphorylation of extracellular signal regulated kinase (ERK) when administered in vitro includes: contacting the cells with the compound, incubating the cells, quantitating phosphrylated ERK and comparing the amount of phosphorylated ERK with total ERK.

59. The method of claim 46, wherein the determining of anti-apoptotic effect includes: contacting osteoblasts or osteocytes from the host with a proapoptotic agent both alone and incombination with the compound; and measuring the inhibition of apoptosis; and comparing apoptosis where a decrease in apoptosis the presence of compound indicates an anti-apoptotic effect.

60. The method of claim 46, further comprising determining the compound does not induce estrous and does not induce significant androgenic gene transcriptional activity.

61. A method of screening for a compound that increases bone mass in a host at least 10% without a loss in bone strength or quality, comprising:
(a) determining that the compound includes the following characteristics:
(i) binds to an androgen like receptor with an association constant of at least 10⁸ M⁻¹ ;
(ii)(a) induces androgenic gene transcriptional activity at a level that is no greater than 10% that of testosterone when administered in vivo at concentrations of 10⁻¹¹ to 10⁻⁷ M at dosage of at least 0.1 ng/kg body weight or in vitro in osteoblastic or osteocytic cells with androgen receptors or (b) induces an increase in muscle weight or virilization in the host of no more than 10% that of testosterone and analogs thereof;
(iii) induces the phosphorylation of extracellular signal regulated kinase (ERK) when administered in vivo at a dosage of at least 0.1 ng/kg body weight or in vitro at concentrations of 10⁻¹¹ to 10⁻⁷ M in osteoblastic cells of the host with androgen receptors; and
(iv) has an anti-apoptotic effect on osteoblasts at an in vivo dosage of at least 0.1 ng/kg body weight or in vitro in osteoblastic or osteocytic cells from the host with androgen receptors; and
(b) selecting said compound having said characteristics.

62. The method of claim 61, wherein determining the association constant includes contacting the compound with an androgen receptor of the host and measure the association constant.

63. The method of claim 61, wherein the association constant is measured by a competitive radiometric binding assay, comprising:
a) contacting the compound with a sample from the host comprising androgen receptors and [³H] synthetic androgen RU1881;
b) incubating to allow binding of the compound to the receptor;
c) isolating bound receptor; and
d) comparing the amount of bound receptor displaced by the compound to a control sample of androgen receptors with [³H] synthetic androgen RU1881 to determine the association constant.

64. The method of claim 61, wherein determining that the compound induces androgenic gene transcriptional activity in vivo comprises: administering the compound to a host and monitoring the level of induction or suppression of a surrogate marker of androgenic transcriptional activity.

65. The method of claim 64, wherein the surrogate marker is prostate specific antigen.

66. The method of claim 61, wherein determining that the compound induces androgenic gene transcriptional activity in vitro comprises: contacting the compound with a cell of the host having androgen receptors and monitoring the level of induction or suppression of a surrogate marker.

67. The method of claim 66, wherein the androgen induced transcriptional activity is assessed in cells selected from the group consisting of: osteoblastic cells with natural androgen receptors, osteocytic cells with natural androgen receptors, traf calvaria cells, MLO-Y4 osteocytic cells and HeLa cells.

68. The method of claim 66, wherein the surrogate marker is prostate specific antigen.

69. The method of claim 61, wherein the phosphorylation is assessed in biopsies from the host using immunohistostaining with specific antibodies against phosphorylated ERKs.

70. The method of claim 69, wherein the biopsy is from bone.

71. A method of screening for compounds that possess bone anabolic effects, comprising:
a) contacting a sample of osteoblast cells with a compound; and
b) comparing the number of osteoblast cells undergoing apoptosis in the compound-treated cells with the number of osteoblast cells undergoing apoptosis in an untreated same of osteoblast cells,
wherein a lower number of apoptotic cells following contact with the compound indicated that the compound possesses bone anabolic effects.

72. The method of claim 71, wherein apoptosis is determined by nuclear morphologic criteria, DNA end-labeling, DNA fragmentation analysis or immunohistochemical analysis.

73. A method of screening for compounds that possess bone anabolic effects, comprising:
a) contacting a sample of osteocyte cells with a compound; and
b) comparing the number of osteocyte cells undergoing apoptosis in the compound-treated cells with the number of osteocyte cells undergoing apoptosis in an untreated same of osteocyte cells,
wherein a lower number of apoptotic cells following contact with the compound indicated that the compound possesses bone anabolic effects.

74. The method of claim 73, wherein apoptosis is determined by nuclear morphologic criteria, DNA end-labeling, DNA fragmentation analysis or immunohistochemical analysis.
